(19) United States
(12) Patent Application Publication
(10) Pub. No.: US 2015/0218625 A1
(43) Pub. Date: Aug. 6, 2015

(54) COMBINATION OF DSDNA BINDING DYE AND PROBES FOR CHARACTERIZATION OF SSDNA SEQUENCES

(71) Applicant: BRANDEIS UNIVERSITY, Waltham, MA (US)

(72) Inventors: John E. Rice, Quincy, MA (US); Yanwei Jia, Waltham, MA (US); Lawrence J. Wangh, Auburndale, MA (US)

(73) Assignee: Brandeis University, Waltham, MA (US)

(21) Appl. No.: 14/428,864

(22) PCT Filed: Sep. 17, 2013
(86) PCT No.: PCT/US2013/060029

§ 371 Date: Mar. 17, 2015

Related U.S. Application Data
(60) Provisional application No. 61/702,019, filed on Sep. 17, 2012.

(51) Int. Cl. C12Q 1/68

(52) U.S. Cl. CPC C12Q 1/6818

(57) ABSTRACT

This invention includes methods for analyzing single-stranded nucleic acid sequences, either RNA sequences or DNA sequences (ssDNA) utilizing dyes that fluoresce when associated with double strands, so-called DNA binding dyes or dsDNA-dyes. Methods according to this invention utilize a dsDNA-dye in combination with one or more hybridization probes that hybridize to a target nucleic acid sequence and that are labeled with a non-fluorescent quencher moiety, for example, a Black Hole quencher.

### CROSS REFERENCE TO RELATED APPLICATIONS

The present application claims priority to U.S. Provisional Patent Application 61/702,019, filed Sep. 17, 2012, which is incorporated by reference in its entirety.

This invention relates to fluorescence detection methods for nucleic acid sequences and to kits for performing such methods.

### BACKGROUND OF THE INVENTION

Detection and analysis of single-stranded nucleic acid target sequences may include the use of fluorescently labeled oligonucleotide hybridization probes, primers, or both. Detection may or may not be “homogeneous detection”. Homogeneous detection means detection that does not require separation of bound (hybridized to target) primers or probes from unbound primers or probes. Among probes suitable for homogeneous detection are linear probes labeled on one end with a fluorophore and on the other end with a quencher, most often a non-fluorescent quencher (5′ exonuclease probes described in, for example, Livak et al. (1995) PCR Methods Appl. 4:357-362), hairpin probes labeled on one end with a fluorescent moiety such as a fluorophore and on the other end with a quencher (molecular beacon probes described in, for example, Tyagi et al. (1996) Nature Biotechnology 14:303-308), double-stranded probes having a fluorophore on one strand and a quencher on the other strand (yin-yang probes described in, for example, Li et al. (2002) Nucl. Acids Res. 30, No. 2 e5), linear probes having a fluorophore that absorbs emission from a fluorescent dye and reemits at a longer wavelength (probes described in, for example, United States published patent application US2002/0110450), and pairs of linear probes, one labeled with a donor fluorophore and one labeled with a quencher or an acceptor fluorophore that hybridize near to one another on a target strand such that their labels interact. A label pair such as a fluorophore and quencher may interact by FRET (FRET probe pairs described in, for example, U.S. Pat. No. 6,140,054). As an alternative to FRET quenching, molecular beacon probes, 5′ exonuclease probes and yin-yang probes may utilize contact quenching, which does not require substantial spectral overlap between the fluorescent moiety's emission spectrum and the quencher's absorption spectrum. Tyagi et al. (1998) Nature Biotechnology 16: 49-53; European Patent EP 0 892 808. Published international patent application WO 2011/050173 discloses analysis of a single-stranded nucleic acid target sequence by hybridizing to the target sequence multiple probes, including one or multiple “On” probes (fluorophore/quencher dual-labeled probes that signal upon hybridization) and one or more “Off” probes (quencher-labeled probes that upon hybridization quench fluorescence from the “On” probes) and analyzing fluorescence of the “On” probes' fluorophore as a function of temperature, generally by means of a melting curve or annealing curve.

DNA binding dyes (dsDNA-dyes) are dyes that fluoresce when interacting with double-stranded nucleic acids, for example, SYBR® Green. dsDNA-dyes have been used for nucleic acid detection in two ways. The first way is to detect double-strands, whether present alone or in the presence of single strands. This involves stimulating the dye in the presence of double strands and detecting emission from the dye. Detection of double strands by dsDNA-dyes is non-specific; that is, it tells whether or not double-strands are present, but it does not tell the sequence or sequences of the strands. The second is to intercalate a dsDNA-dye into hybrids of fluorescently labeled probes and single-stranded targets, to stimulate the dye at its absorbance wavelength and to detect emission from the probe's fluorescent label at its emission wavelength, wherein the probes' labels are excited by the dye's emission by FRET. Because this involves FRET, the absorption spectrum of the probe's fluorescent moiety must substantially overlap the emission spectrum of the dye. When a dye is used in combination with one or more probes by detecting dye emission as a non-specific indicator of double-strands and by detecting probe emission as an indicator of specific sequences, there is normally a restriction that the probe signal must be detectably distinct from the dye signal. Because the most common dsDNA-dye, SYBR® Green, has essentially the same emission spectrum as the most common fluorescent moiety for probes, the fluorophore FAM (See FIG. 1 of Van Poucke et al. (2012) BioTechniques 52: 81-85), the two cannot normally be used together. See additionally Lind et al. (2006) BioTechniques 40:315-318, who report that FAM has an excitation maximum at 493 nm and an emission maximum at 525 nm, and SYBR® Green has an excitation maximum at 497 nm and an emission maximum at 516 nm. Lind et al. addressed this problem by using a FAM-labeled probe, not with SYBR Green, but with a different dsDNA-dye that is distinguishable from FAM, namely, BEBO, which has an excitation maximum at 515 nm and an emission maximum at 552 nm. Van Poucke et al. reported a TaqMan® (5′ nuclease) assay, a symmetric PCR assay that generates probe fragments carrying a fluorophore label, wherein the probes' label was FAM and wherein SYBR® Green was used to detect that an amplicon was made, in this case a double-stranded amplicon. A melting peak was obtained at about 82° C. Because SYBR signal predominated, that indicated melting of double strands, a non-specific indication that some product had been made. By then detecting at a higher temperature at which the double strands were melted, 85° C., the authors concluded that fluorescence above that resulting from a no-template control (NTC) was from probe fragments. Supporting that conclusion was the fact that there was no high-temperature melting peak, and constantly fluorescing probe fragments would not produce a melting peak. Of course, there was no detection of probe-target hybrids, because they would have melted off at a temperature below the melting temperature of double-stranded amplicon. Neither Lind et al. nor Van Poucke et al. utilized the SYBR® Green dsDNA-dye for analysis of single strands. Van Poucke et al. did not use FAM for any analysis of probe binding as a function of temperature.

### SUMMARY

This invention includes methods for analyzing single-stranded nucleic acid sequences, either RNA sequences or DNA sequences (ssDNA) utilizing dyes that fluoresce when associated with double strands, so-called DNA binding dyes or dsDNA-dyes. Methods according to this invention utilize a dsDNA-dye in combination with one or more hybridization probes that hybridize to a target nucleic acid sequence and that are labeled with a non-fluorescent quencher moiety, for example, a Black Hole quencher. Such probes are generally referred to herein as “quencher-labeled probes.” Methods according to this invention comprise analyzing fluorescence from a dsDNA binding dye as a function of temperature over a temperature range that includes the melting temperature of such hybridization probe or probes. A target sequence may be a sequence to which the probe or probes are perfectly complementary or less than perfectly complementary. In certain embodiments, quencher-labeled probes can be “in situ probes,” as explained below.

For use with a dsDNA-dye in methods of this invention a quencher-labeled probe or a probe set containing at least one non-fluorescent-quencher-labeled probe can be used to detect and analyze related sequences to which they hybridize. Probe sets useful in methods of this invention may include additionally a probe or probes that include a fluorescent-quencher label or that do not include any quencher label.

dsDNA-binding dyes in general are useful in methods of this invention. The dsDNA-binding dye most commonly used is SYBR® Green dye. Other dsDNA-dyes include ethidium bromide, DAPI, BO and BEBO (Bengtsson et al. (2003) Nucleic Acids Research 31:e45). Yet another is BOXTO (Lind et al. (2006) BioTechniques 40:315-318). At least some of these dyes are reported to be minor groove binding dyes. In methods of this invention dsDNA-dyes may be used alone or in combination.

Hybridization probes useful in methods of this invention include both linear, or random-coil, probes and structured probes. They may be RNA, DNA or combinations of RNA and DNA. They may include non-natural nucleotides, nucleotide analogs and non-natural inter-nucleotide linkages. Non-natural nucleotides and analogs that increase the binding affinity of probes include, for example, 2′-O-methyl ribonucleotides and PNA. Structured probes may comprise one strand (for example, molecular beacon probes) or two strands (for example, yin-yang probes). Certain embodiments of this invention utilize probes that initially comprise primer extensions and whose construction is completed in situ as part of post-amplification detection. For convenience we refer to such probes as “in-situ probes.”

Hybridization probes useful in methods of this invention include unlabeled probes, or unlabeled oligonucleotides, and labeled probes. Labels are categorized with regard to the dsDNA-dye with which the probes are used. To obtain dye emissions one excites a sample with light having a wavelength at or near the absorption maximum of the dye, and emissions are detected at or near the maximum emission wavelength of the dye. This is commonly referred to as reading in the dye channel of an instrument or, if the dye is SYBR® Green, reading in the SYBR® Green channel. As an example, for SYBR® Green dye, a typical excitation is at 470 nm, for example by a blue LED, and detection is made using a 510-nm emission filter. A first category of labels is “dye-quenching labels,” that is, labels that quench dye fluorescence when the probe is hybridized to its target, forming a double-stranded region, and the dye is excited at or near its maximum absorption wavelength. We believe that dye quenching is by fluorescence resonance energy transfer (FRET), and for convenience refer to it herein as FRET quenching. In general, or FRET to occur there should be significant overlap between the emission spectrum of the dye and the absorption spectrum of the label. A dye-quenching label may be a non-fluorescent quencher, for example DABCYL, a Black Hole quencher, a QSY quencher, an Eclipse quencher, a Deep Dark quencher, an Iowa Black quencher or a Blackberry quencher. If a non-fluorescent quencher is highly efficient, for example, a Black Hole Quencher 1 or a Black Hole Quencher 2, a single quencher label will suffice. If the quencher is less efficient, for example DABCYL, at least two quencher labels may be desired or even necessary. Probes with a single quencher moiety are simpler and less expensive to synthesize than dual-labeled probes. In-situ probes, as finally constructed, can include a single non-fluorescent quencher or no label. Alternatively, a dye-quenching label may be a fluorescent moiety, for example a fluorophore or a Quantum Dot, that is not excited directly at the excitation wavelength used to excite the dye but rather accepts energy from the dye by FRET, thereby quenching dye emission, and remits fluorescence at a wavelength different from (longer than) the wavelength at which the dye's emission is detected. For example, the fluorophore Cal Orange accepts energy from the dye SYBR® Green. Such a label is stimulated indirectly by exciting the ds-DNA dye, but its fluorescence is acquired, not at a wavelength at or near the maximum emission of the dye, but rather at a longer wavelength at or near the emission maximum of the fluorescent label. When emissions are detected at or near the wavelength of maximum dye emission, fluorescence emission from the label is not detected, and its impact is simply quenching of the dye. Fluorescent labels of this category are known for use in ResonSense® probes. A ResonSense® probe is a single-stranded oligonucleotide labeled with a fluorophore that accepts fluorescence from the dsDNA-dye. When a sample containing a hybridized ResonSense® probe is illuminated at the absorption wavelength of the dsDNA-dye, the label absorbs energy from the dye by FRET and reemits light at a longer wavelength equivalent to the emission spectrum of the fluorophore. The fluorophore is one that is spectrally distinct from the dye. When a probe so labeled is not hybridized, excitation of the dye does not indirectly excite the fluorophore, so the fluorophore need not be quenched. However, if it desired to stimulate the fluorophore directly, a probe with a dye-quenching fluorescent label is dual-labeled and of a construction that causes it to be quenched when unhybridized but to be unquenched and to emit a detectable signal when hybridized, for example, a quenched ResonSense® probe, a molecular beacon probe or a yin-yang probe. A second category of labels is labels that are excited by the wavelength of light used to excite the dye. An example is the fluorophore FAM when used with SYBR® Green dye. FAM has a maximum excitation wavelength of 493 nm and a maximum emission wavelength of 525 nm, whereas SYBR® Green has a maximum excitation wavelength of 497 nm and a maximum emission wavelength of 521 nm. Excitation of the dye also excites the label, and detection at an emission wavelength appropriate for the dye detects emissions from both the dye and the label. A label of this category results in an emission spectrum that is different from the emission spectrum (both as a melting curve and first derivative curve) that would be obtained from the dye alone in the absence of the label. We refer to labels of this category as “dye-coincident labels.” A probe with a dye-coincident label is dual-labeled and of a construction that causes it to be quenched when unhybridized but to be unquenched and to emit a detectable signal when hybridized, for example, a molecular beacon probe or a yin-yang probe. A third category of labels are labels that neither are excited by the light used to excite the dye nor are capable of accepting energy from the dye by FRET, because their absorption spectra overlap neither the absorption spectrum nor the emission spectrum of the dye. An example is the fluorophore Alexa Fluor 790 when used with the dye DAPI. Alexa Fluor 790 absorbs at a wavelength longer than DAPI's emission, so excitation of the dye does not excite the label either directly or indirectly. Labels in this category are irrelevant insofar as concerns excitation and detection at wavelengths appropriate for the dye, but they can be used to gain information by separate excitation and detection. We refer to labels of this category as “non-overlapping labels.” A probe with a non-overlapping label is dual-labeled and of a construction that causes it be quenched when unhybridized but to be unquenched and to emit a detectable signal when hybridized, for example, a molecular beacon probe or a yin-yang probe.

In this specification we refer to melting temperatures (“Tms”) of primers and probes. “Melting temperature” is the temperature at which a nucleic acid hybrid, for example, a probe-target hybrid or primer-target hybrid, is 50% double-stranded and 50% single-stranded. It will be appreciated that the Tm of a probe relative to a more complementary target sequence is higher than the Tm of that probe relative to a less complementary target sequence containing a deletion, an addition, or one or more mismatched nucleotides For a particular assay the relevant Tm's may be measured. Tm's may also be estimated by calculation utilizing known techniques. For this purpose we utilize Tm_{[0]}, a calculated Tm utilizing the “nearest neighbor” method (Santa Lucia, J. (1998), PNAS (USA) 95: 1460-1465; and Allawi, H. T. and Santa Lucia, J. (1997), Biochem. 36: 10581-10594). For labeled probes, both linear and structured, it will be understood that the calculated Tm_{[0]} is an approximation. For instance, the data obtained in Example 1 shows that labeling a probe with a Black Hole Quencher 1 reduces the actual Tm of the probe by about 5° C. “In-situ probes,” described more fully below, have both an initial Tm and a final Tm. The initial Tm is the temperature at which 50% of the 3′ ends of the initial probe sequence in the primer extension touch down on the target sequence within the same strand. Following extension in situ to create the final probe, the initial Tm of the hybrid is replaced by the higher final Tm.

Methods of this invention utilize probe sets, by which we mean one probe or multiple probes that hybridize to a target sequence. A single probe, including a single in-situ probe, may be utilized for detection of a single nucleotide polymorphism (“SNP”). In such an embodiment the probe is a dye-quenching probe whose complementary sequence in the target includes the SNP. When a probe set for a particular target sequence comprises a single probe, the probe includes a non-fluorescent quencher label. An example of such an embodiment is illustrated by Example 1, wherein a single probe is labeled with either one or two Black Hole quenchers. A single dye-quenching probe can also be multiply labeled with a non-fluorescent dye-quenching label and a fluorescent dye-quenching label, a dye-coincident label or a non-overlapping label. We normally design a single probe to be more complementary to a wild-type or drug-sensitive variant of the target sequence, and less complementary to mutant or drug-resistant variants. In certain embodiments, including some embodiments utilizing in-situ probes, we do the reverse. In the case of self-reporting in-situ probe, it is generally desirable to design the 3′end of the single-strand to be more perfectly complementary to one sequence variant, for instance the wild-type version of the sequence, and less perfectly complementary to other versions, SNPs, within the sequence to which the 3′ end initially hybridizes.

When a probe set contains multiple probes, the probe set includes at least one dye-quenching probe labeled at least with a non-fluorescent quencher, as described in the preceding paragraph. A dye-quenching probe may be an in-situ probe. Preferred multiple-probe sets include two or more of such dye-quenching probes. An example of such an embodiment is illustrated by Example 2, wherein the probe set includes six probes, each singly labeled with a Black Hole quencher. Here again, such probes in multi-probe sets can be multiply labeled with a dye-quenching fluorescent label (see Example 5), a dye-coincident label (see Example 3) or a non-overlapping label. In some embodiments a set of multiple probes can include one or more unlabeled probes or one or more probes that are labeled only with a fluorescent dye-quenching label. An in-situ probe, for example, may be unlabeled in certain embodiments. In the case of multi-probe sets it is required that there be sufficient dye quenching to provide meaningful distinguishing information regarding the target sequence be obtained when a sample is excited at or near the maximum absorption wavelength of the dsDNA-dye and detection is at or near the maximum emission wavelength of the dye, as is explained below in connection with Example 5.

Principal criteria that are utilized to design a multi-probe set are the temperature range that is to be utilized for detection and the length of the target sequence to which the set hybridizes. The temperature range is below the Tm of double-stranded amplicons, which determines an upper limit. The lower limit is a chosen temperature within the capability of the detection instrument, usually at least as high as room temperature. With a temperature range selected, the probe Tm's are designed to be in the range. To fit a given type of probe, say a DNA probe, in the range, one can adjust the length of the probe or its degree of complementarity to the target-sequence variants, or both. When probes of a multi-probe set hybridize to their single-stranded nucleic acid target sequence, the probes spread along the target sequence so as to pick up sequence variations. They may hybridize directly next to one another, there may be modest overlap of adjacent probes, or there may be gaps of one, a few, or even many nucleotides between adjacently hybridized probes. We prefer that gaps be minimized Probes in a probe set may have, and generally do have, different probe-target Tm's, which permits variations in fluorescent contours and fluorescent signatures to occur at multiple temperatures in the detection range. Probes Tm's differing by at least 2° C. will change the fluorescence signature by altering the shape of a valley (or peak). Probes whose Tm's differ by 5° C. or more advantageously lead to separate melting or annealing peaks. However, certain embodiments may include two probes having the same or almost the same Tm, which we refer to as “Tm stacking,” in which case both probes will contribute to a single melting or annealing peak, for example see FIG. 5, line 507. In designing a probe set for a LATE-PCR amplification reaction, we prefer that the maximum probe Tm against all target sequence variants be below the annealing temperature used in the amplification reaction, which generally is not higher than about 80° C.

Methods of this invention include use of multiple probe sets in the same detection mixture, that is, a probe set for each of at least two target sequences. The probe sets may be distinguishable by Tm. For example, a probe set for a first target sequence may include probes having Tms in a range of 55-70° C., and a probe set for a second target sequence may include probes having Tms in a range of 40-54° C. Alternately, two probe sets may have overlapping Tms. See Example 3, in which a probe set for a 16s-gene target sequence is used in a single detection mixture with a probe set for a gyrase B-gene target sequence, and each probe set, when used alone, produced (negative) melting peaks near 50° C. Nonetheless, when the sets were used together, combined detection has been shown to be capable of yielding strain-distinguishing information.

In-situ probes-can be used in combination with additional quencher-only probes or dual-labeled probes. Sequence-specific probes are particularly useful in such sets because they selectively hybridize to, or selectively fail to hybridize to, particular SNP's. Formation of such a probe-target hybrid significantly reduces the flexibility of said single strand and thus inhibits formation of a self-reporting in-situ probe. The Tm of the sequence-specific probe is designed to be higher, for instance 60° C., than the Tm of the initial Tm of the 3′end to its target, say 45° C. Upon extension of the 3′ end of the final-Tm of the 3′end will increase and can become equal to or higher than the Tm of the sequence-specific probe.

Methods of this invention include contacting, under hybridizing conditions, a sample that includes copies of at least one single-stranded oligonucleotide that is a nucleic acid strand containing at least one target sequence with a dsDNA-dye and a probe set for the at least one target sequence, so as to hybridize the probes in the set to the target sequence or sequences. Such a nucleic acid strand may be DNA or RNA. It may contain a single target sequence, in which case the target sequence may comprise essentially the entire strand, or it may contain at least two target sequences, in which case the strand is sufficiently long to encompass all target sequences. Copies of the single-stranded nucleic acid target sequence can be provided by any means. Single-stranded nucleic acid target sequences for analysis can in some instances be obtained directly by isolation and purification of nucleic acid target strands in a sample. For example, DNA plus strands containing target sequences can be obtained from samples containing double-stranded DNA by separating plus and minus DNA strands and isolating target-sequence containing strands, for example the plus strands, by removing the complementary strands, for example the minus strands. Some embodiments of methods according to this invention include nucleic acid amplification. If the amplification is symmetric, the amplification product or products are double-stranded, and copies of the at least one target strand can be obtained by separating plus and minus strands, as described above. Numerous known amplification methods can be used, including methods that employ the polymerase chain reaction (PCR), NASBA, SDA, and rolling circle amplification. For example a symmetric PCR method may include labeling one primer with biotin and separating the biotin-containing product strands from the other strands by capture onto an avidin-containing surface, which is then washed. Amplification can start with double-stranded nucleic acid strands that contain a target sequence, or amplification can start with single strands that contain a target sequence or a sequence complementary to a target sequence. Amplification may include reverse transcription followed by amplification of cDNA, where the starting sample is RNA. The above methods can be performed in a microfluidics device, which may permit, for example, the inclusion of wash steps. For use of dsDNA-dye in a microfluidics device, methods of this invention include adding to the source of dye a double-stranded carrier to overcome the problem of the dye sticking to the walls of the device.

In certain preferred methods, copies of the single-stranded target sequence are provided by non-symmetric nucleic acid amplification. Single-stranded deoxyribonucleotide (DNA) target sequences for analysis, either conserved sequences or variable sequences, can be obtained by non-symmetric amplification methods that utilize one primer in a limiting amount such that it runs out during the amplification reaction (the limiting primer), after which single-stranded amplification product continues to be produced utilizing the other primer (the excess primer). Most commonly used non-symmetric amplification methods are PCR methods, including asymmetric PCR and LATE-PCR, either of which can be combined with reverse transcription for starting with an RNA sequence. Our preferred amplification method is LATE-PCR. Non-symmetric amplification methods generate both double-stranded amplification products, or “amplicons”, and single-stranded amplification products (amplicons) that contain target sequences to be analyzed.

Presence of double strands in the sample during fluorescence acquisition has implications for design of the detection method. When double strands are present with single strands in a sample being analyzed, the dsDNA-dye is added in an amount sufficient to bind not only to the double strands, but also to the relatively short probe-target hybrids. If the sample contains only single-stranded nucleic acids, probe Tm's are not constrained. They can range from very low, generally room temperature, to very high, 90° C. for example. However, if signal-producing double strands are also present during fluorescence acquisition, for example, double-stranded amplification products (or double-stranded “amplicons”), probe Tm's are kept below the Tm or Tm's of the signal-producing double strands.

Methods of this invention include stimulating a sample containing at least one target, dye and a probe set for the at least one target with light of a wavelength at or near the dye's absorption maximum and detecting emissions at or near the dye's emission maximum over a temperature range that includes the Tms of the probes. Considering a single target sequence-containing strand, which in certain embodiments is a single-stranded amplicon, the highest Tm will be the melting temperature of the strand itself if hybridized to its complementary sequence, or complement. Probes in a probe set, being shorter than the entire strand, will have lower Tm's, so detection of probe melting takes place at temperatures below the strand Tm. The temperature range for detection preferably includes the Tm of double-stranded target, if present. The step of detecting may be performed as a melt, in which the temperature is lowered to the bottom of the range and then progressively increased over time to the top of the range with frequent acquisition of emissions to generate a fluorescent contour, namely, a melting curve. This step may be performed in the opposite manner, in which the temperature is raised to the top of the range and then progressively lowered over time to the bottom of the range with frequent acquisition of emissions to generate an annealing curve. In some cases, it may be desirable to carry out two cycles of melting or annealing. For instance, when in-situ probes are used with sequence-specific probes the first cycle of melting can result in 3′end extension, thereby increasing the initial Tm to the final-Tm. This change, in turn, can alter the level of sequence-specific probe hybridization. This change in Tm can be observed by using two rounds of annealing or melting of probe-target hybrids.

In some embodiments a complete melting curve or annealing curve can be replaced by fluorescence acquisition at only a few selected temperatures, in some cases only a single selected temperature. If one or more of the probes contains a non-overlapping label, the method may include separately stimulating that label directly with light of an appropriate wavelength, and detecting emission at or near the label's emission maximum as a function of temperature or as a function of cycle number for real-time detection, or both. If one or more of the probes contains a fluorescent dye-quenching label, the method may include stimulating that label either directly or indirectly (by exciting the dye) and detecting emission from the label as a function of temperature or as a function of cycle number for real-time detection, or both. Detecting is normally done at a relatively narrow wavelength range that, for example, acquires fluorescence only from the dsDNA-dye and a dye-coincident label and excludes fluorescence from other fluorophores. However, detecting at a broad wavelength range that acquires fluorescence from the dye, dye-coincident label and at least one additional fluorophore is not excluded.

Methods of this invention include a step of comparing the emission as a function of temperature at the dye's wavelength from the sample containing the target nucleic acid sequence to at least one corresponding emission for a reference sample of known composition, generally a sample of known sequence. Melting curves can be compared, as can annealing curves. Readings at select temperatures can be compared to readings at select temperatures. When detection includes obtaining a temperature-dependent fluorescent contour, measured either as a melting fluorescent contour or an annealing fluorescent contour, the fluorescent contour can be mathematically converted into a fluorescent signature, which is the first derivative of the fluorescent contour. Comparison with a reference can be comparison of fluorescent signatures. The lowest value of each negative peak below background, which we sometimes refer to as a “valley”, or the total area of each valley below background, is proportional to the amount of the target present initially in the sample. A fluorescent signature can be further converted into a normalized fluorescent signature by dividing all values of the fluorescent signature by the lowest valley or the highest positive peak value, if there is a fluorescent label that contributes a fluorescent peak. Comparison with a reference can be comparison of normalized fluorescent signatures. Another comparison useful in methods of this invention is a ratio of fluorescence at two selected temperatures, wherein the ratio of a sample containing the target is compared to the ratio of a reference. Comparisons can be done by analyzing a sample and one or more references in parallel. Alternatively, comparison can be to a library of fluorescent signatures or other results obtained from reference samples.

Methods according to this invention may be used, for example, to analyze a sample for the presence of a human or pathogen mutation, or to screen a sample to detect which one of multiple possible target species is present. Methods of this invention may include multiplex detection; that is, probe set may be included for more than one target.

This invention also includes reagent kits for performing analytical methods according to this invention that include non-symmetric nucleic acid amplification to supply ssDNA target sequences for analysis. Such kits include, for each target, at least a limiting primer and an excess primer, dNTPs, dsDNA-dye, at least one probe set as described above, and DNA polymerase.

### BRIEF DESCRIPTION OF THE DRAWINGS

FIGS. 1A-1D are fluorescent signatures (first derivative of dsDNA-dye fluorescence intensity versus temperature, in this case as temperature is increased) for a variable single-stranded target region of a gene of two strains of M. tuberculosis using either no probe (FIG. 1A) or one of three versions of a DNA hybridization probe: one unlabeled (FIG. 1B), a second labeled with a single non-fluorescent dye-quenching moiety (FIG. 1C), and a third labeled with two non-fluorescent dye-quenchers (FIG. 1D).

FIG. 2A is fluorescent signatures for a variable single-stranded region of a gene of several strains of M. tuberculosis using a probe set consisting of six different DNA dye-quencher-labeled hybridization probes, each an Off probe labeled only with a single non-fluorescent quencher.

FIG. 2B is a plot of the average minimum fluorescent rate value of the probes to the various targets shown in FIG. 2A versus the corresponding threshold thermal cycles (C_{T}) at which the signal was detected in the dsDNA-dye channel.

FIG. 3A-3C are fluorescent signatures from a multiplex LATE-PCR amplification of variable regions of two genes of six Mycobacterium species, wherein the reaction mixtures contained a probe set for one gene (FIG. 3A), a probe set for the other gene (FIG. 3B), or both probe sets (FIG. 3C). One probe set consisted of two probes: one dual-labeled with a non-fluorescent dye-quenching moiety and a dye-coincident fluorophore that was not spectrally distinct from the dsDNA-dye (an On probe), and one singly labeled with a non-fluorescent dye-quenching moiety (an Off probe). The other probe set consisted of four probes: one unlabeled and three dye-quencher-labeled. Of those three, one was singly labeled with a non-fluorescent dye-quenching moiety (an Off probe) and two were dual-labeled with a non-fluorescent dye-quenching moiety and a dye-coincident fluorophore that was not spectrally distinct from the dsDNA-dye (On probes).

FIG. 4A-4C are fluorescent signatures from a multiplex LATE-PCR amplification as for FIG. 3 for two species using the two-probe set, wherein the On probe was the same as in Example 3, but the Off probe was singly labeled with an efficient non-fluorescent dye-quenching label (FIG. 4C) or singly labeled with a less-efficient quencher (FIG. 4A) or doubly labeled with the less-efficient quencher (FIG. 4B).

FIG. 5A-5C are fluorescent signatures in the dye channel (5A, 5C, 5E, 5G) and in the Quasar 670 channel (5B, 5D, 5F, 5H) from a LATE-PCR amplification of a genetic target sequence of two bacterial species wherein the probe set initially comprised three singly labeled Off probe and three dual-labeled On probes having non-overlapping fluorophores, and wherein progressively one, then two, then three On probes were replaced by unlabeled probes.

FIG. 6 is a schematic depiction of the microfluidics device used in Example 6.

FIG. 7A-7D is a series of photomicrographs showing spots of fluorescence obtained from various samples in Example 6 with the device shown in FIG. 6, demonstrating the ability of dsDNA to transport dsDNA-dye.

FIGS. 8A-8D are schematic representations of how in-situ probes are designed, how they form, and how their formation is impacted by the presence of a bound sequence-specific probe.

### DETAILED DESCRIPTION

While wishing not to be bound by any theory, we believe, based significantly on the work reported in the Examples, that a fluorescent contour of a closed-tube reaction that contains multiple fluorescent or quenching components provides an instantaneous or near instantaneous temperature-dependent description of all of the components of the system (whether an oligonucleotide or a dsDNA-dye), including those whose fluorescence or absorption increases as a function of temperature and those whose fluorescence or absorption decreases as a function of temperature, as well as all molecular interactions of all such fluorescent and non-fluorescent components in the system including, for instance, the proportion of each dye-quenching probe that is hybridized to or not-hybridized to a possible target at a particular temperature. Although we sometimes refer to the first derivative of a “fluorescent contour,” that is, a graph of fluorescence intensity versus temperature, in such a system as a melt curve or annealing curve, it is not a traditional melt curve or annealing curve whose peak traditionally defines the melting temperature, Tm value, of a probe/target hybrid. Rather, in methods of this invention a fluorescent contour and its first derivative, a “fluorescent signature,” describe the sum of numerous temperature-dependent dynamic equilibriums among all of the reaction components in all of their possible conformations, as well as all interactions of these components. It is nonetheless surprising that alteration of a single component, such as the sequence of an amplified target, leads to a significantly detectable change in the fluorescent signature obtained from a dsDNA-dye.

In methods of this invention a dsDNA-dye is included in reaction mixture during fluorescence acquisition. Determining an optimum amount of dye in a particular embodiment is readily done by simple trial and error. In the Examples we performed LATE-PCR amplifications in which SYBR® Green concentrations of 0.24× and 0.48× were satisfactory, but a concentration of 0.72× prevented amplification (complete inhibition). We also performed LATE-PCR reactions using a microfluidics device having fill channels of PDMS, to which SYBR® Green dye is known to stick. In that case we included in the reaction mixture a source of double strands that carried sufficient dye into the reaction mixture during amplification when the initial SYBR® Green concentration was 0.96×.

Probe sets used in methods of this invention include use of at least one labeled hybridization probe. Hybridization probes useful in methods of this invention include both linear, or random-coil, probes and structured probes. They also include in-situ probes. They may be RNA, DNA or combinations of RNA and DNA. They may include non-natural nucleotides, nucleotide analogs and non-natural inter-nucleotide linkages, for example, PNA probes or 2′-O-methyl ribonucleotides. They may be linear (random coil) or structured, for example LNA probes. Structured probes may comprise one strand (for example, molecular beacon probes) or two strands (for example, a yin-yang oligonucleotide structure). Additional probe types that may be used include Light Cycler probes and minor groove binding probes. Several such probes are described in WO 20111/050173A1, where they are referred to as signaling probes.

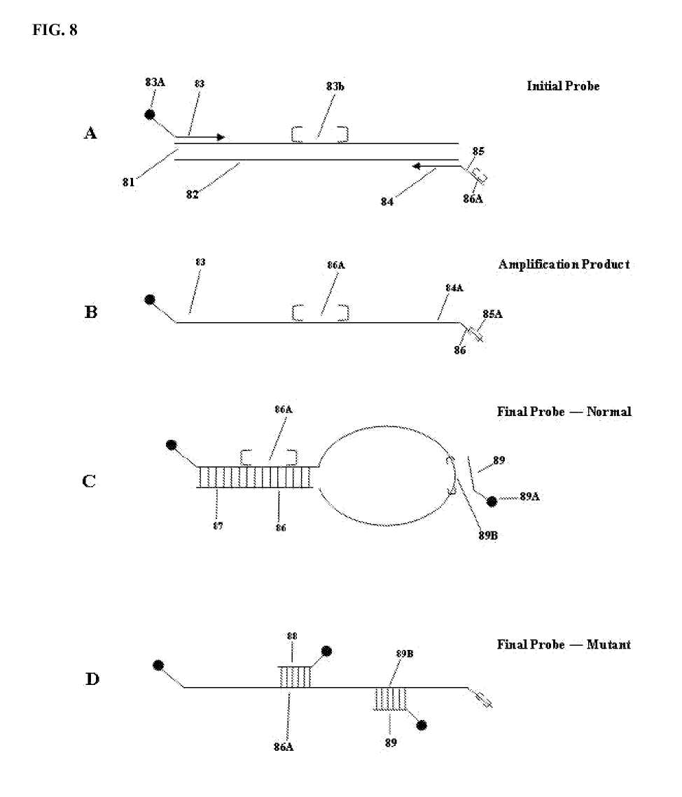

Structured probes useful in methods of this invention also include “in-situ probes.” Construction and use of in-situ probes is shown schematically in FIGS. 8A-8D. The initial form of an in-situ probe for use with non-symmetric amplification, preferably LATE-PCR amplification, is shown in FIG. 8A in conjunction with double-stranded target sequence 81, 82. Initially the probe comprises excess primer 83, limiting primer 84 and limiting-primer extension 85. In certain embodiments, excess primer 83 includes a 5′-terminal dsDNA quenching moiety, preferably a Black Hole quencher or other efficient non-fluorescent quencher. Also shown in FIG. 8A is probing site complement 86, which differs among variants of the target sequence, for example, between a drug-sensitive bacterial strain and a mutant, drug-resistant strain. Limiting-primer extension 85 includes probing sequence 86A, which is complementary to one variant of sequence 86 (complementarity may be, but need not be, perfect) but mismatched (or more mismatched) to the other variant or variants. It will be appreciated that limiting-primer extension 85 is designed not to form a primer-dimer with excess primer 83. FIG. 8B shows the single-stranded amplification product (the excess primer strand), which contains excess primer 83, the complement 84A of the limiting primer, and the complement 85A of the limiting-primer extension, including probing sequence 86. Also shown in FIG. 8B is probing site 86A. The limiting-primer extension's probing site complement 86A is designed such that the Tm of the hybrid 86, 86A is at least 5° C., preferably at least 10° C., below the primer annealing temperature during amplification. Under these conditions the 3′end of the excess primer strand does not bind to its internal complementary sequence during amplification.

The final in-situ probe construction is shown in FIG. 8C. The 5′ end of the limiting-primer extension serves as a template for synthesis of a sequence complementary to the 3′ end of the excess primer strand. After amplification the temperature of the amplification reaction mixture is lowered below the primer-annealing temperature sufficiently for probing sequence 86 to hybridizes to only the intended variant of sequence 86A and to be extended by DNA polymerase to include sequence 87, which is complementary to excess primer 83, thereby forming a double-stranded stem region that include sequences 83, 86A on one side and sequences 87, 86 on the other side. The Tm of the stem is the final Tm of the in-situ probe. It is determined by the length and sequence composition of the resulting double-stranded region. Thus, there is experimental control over the length of the single-stranded region, the double-stranded region, and the temperature at which the double-stranded region forms. Selective extension to form the stem can be enhanced by inclusion of a reagent such as PrimeSafe™ in the reaction mixture. It will be appreciated that due to extension 87 the stem Tm is higher than the Tm of hybrid 86, 86A. Formation of that stem creates loop 89A. Shown in FIG. 8C is another probe 89 and its probe-binding site 89A, which also differs between or among variants of the target sequence. Probe binding site 89A is in the loop resulting from formation of the in-situ probe.

The in-situ probe shown in FIG. 8C can be formed from either the normal (for example, wild-type) variant of the target sequence or a mutant variant. If the assay is designed to detect a single SNP, for example, we prefer to form the in-situ probe with the mutant sequence and to utilize an excess primer having a quencher 83A, which results in the in-situ probe being a high-temperature Off probe that interacts with dsDNA-dye that associates with the probe's stem. If multiple mutant variants may be present, the limiting-primer (FIG. 8A) can be a mixture of limiting primer having different extensions, one having a sequence 86A for each mutant and, thus, leading to different Tm's among the mutants. For multiplex reactions, detection of different double-stranded amplicons of similar size generated in the same reaction is typically not possible with dsDNA-dye such as SYBR Green® due to the transfer of the dye in favor of the highest Tm amplicon. Methods of this invention utilizing in-situ probes enable multiplex product detection using dsDNA-dye by designing the 5′ terminal sequence on the limiting primers for each different single-stranded amplicon to form its own intra-molecular in-situ probe having a unique Tm and structure.

For this approach, probing sequence 86 is designed to be very allele-specific so that it will not hybridize to the normal target sequence at reasonably low temperature to be used during in-situ probe formation and during detection. Such allele-specific intra-molecular priming and extension can be achieved by methods known in the art for the design of allele-specific primers. See, for example, Tyagi et al. U.S. Pat. No. 6,277,607. In detection by melting at temperatures above the stem-formation temperature, the normal single-strands will not generate a dye signal, because they remain single-stranded. Detection of mutant-specific in-situ probes depends only on the absolute number of mutant targets in the mixture and the sensitivity of detection in the dye channel.

FIG. 8C also shows the final construction of an in-situ probe for the normal sequence when probe-binding sequence 86A in the target sequence is the normal, wild-type or drug-sensitive target-sequence variant. For such an embodiment FIG. 8D shows the mutant or other target-sequence variant not selected for in-situ probe formation. The single-stranded amplification product of that variant does not form a loop, and there is no stem that attracts dsDNA-dye whose fluorescence is affected by quencher 83A. Certain embodiments of this type include the use of at least one dye-quenching probe 88, which is complementary to the non-selected (mutant) variant of sequence 86A (complementarity may be, but need not be, perfect) but mismatched (or more mismatched) to the selected (normal) variant. Probe 88 has a Tm that is below the primer-annealing temperature so as not to adversely affect the efficiency of amplification. In-situ probing sequence 86 has an even lower Tm. Probing sequence 86 has a Tm relative to the normal variant of sequence 86A such that a hybrid forms only when the temperature is lowered sufficiently for essentially all probe 88 hybrids to have already formed. After amplification the temperature of the reaction mixture is first lowered to hybridize probe 88 to probe-binding sequence 86A in mutant target sequences (FIG. 8D). Then the temperature is lowered further to create in-situ probes in normal target sequences (FIG. 8C). The presence of relatively stiff double-stranded hybrid 86A, 88 in strands containing the mutant variant prevents in-situ hybridization in these strands. Due to the presence of that double-stranded region, formation of an in-situ hybrid in mutant strands (FIG. 8C) either is prevented completely due to the physical constraint, or the Tm for in-situ hybridization is lowered significantly. Either way, the temperature window of allele discrimination is effectively increased. In creating embodiments of this type, excess primer 83 may include quencher 83A, or it may be unlabeled. In the latter case, the stem of the final in-situ probe (FIG. 8C) will be unlabeled. FIGS. 8C and 8D also show a second dye-quenching probe 89 that includes dye-quenching label 89A and is complementary to probe-binding sequence 89B, which also varies between normal strands and mutant strands. Like probe 88, probe 89 is designed to have a higher Tm against mutant strands than against normal strands. It will be noted that in mutant strands (FIG. 8D) probe-binding sequence 89B is linear, whereas in normal strands (FIG. 8C) probe binding sequence 89B is in the loop of the in-situ probe. This significantly increases the difference between its Tm versus mutant strands (FIG. 8D) and its Tm versus normal strands (FIG. 8C), thereby increasing the window of allele discrimination for this second probe. The presence of mutant excess primer strands in a mixture of normal and mutant single-stranded products decreases the total amount of the normal intra-molecular in-situ probe formed relative to the amount of the normal intra-molecular in-situ probe made in control samples with an equivalent number of 100% normal excess primer strands. Thus, it is possible to calculate the ratio between the amount of bound mutant-specific probes 88 to mutant excess primer strands (a signal increase in the dye channel) and the relative decrease in the amount of intra-molecular in-situ hybrids on normal excess primer strands. Such a ratio provides an exquisitely sensitive measure of the fraction of mutant targets in the mixture, particularly at low percentages.

Probe sets used in methods of this invention include at least one probe having a dsDNA-dye-quenching label. Preferred dye-quenching labels are non-fluorescent quenchers, more preferably non-fluorescent quenchers that are efficient acceptors of energy from the deDNA-dye that is utilized. Less efficient non-fluorescent quenchers or fluorescent moieties that accept energy from the dsDNA-dye by FRET are less preferred as dye-quenching labels. If a probe has, for example, only a fluorescent quencher for the dye, that is, a linear ResonSense® probe, the label will not be excited at the excitation wavelength used to excite the dye, when the probe is not hybridized; but, when the probe is hybridized, the label will be excited indirectly by excitation of the dye. A probe with a dye-quenching label may include at least one additional label. For example, a non-fluorescent dye-quenching label may be added to a ResonSense® probe, which provides additional dye quenching and also permits fluorescence acquisition at the emission wavelength of fluorescent label by exciting that label directly. A probe with a non-fluorescent dye-quenching label may also include at least one dye-coincident or non-overlapping label, for example a fluorophore or Quantum Dot, and have a structure that causes a detectable change in fluorescent signal from its fluorescent label when it hybridizes to a nucleic acid target sequence. When such a probe is in solution, the fluorescent label is quenched by the non-fluorescent quencher label. That quenching may be achieved by any mechanism, typically by FRET (Fluorescence Resonance Energy Transfer) between a fluorescent moiety, or by contact quenching, which is generally more efficient and, therefore, preferred. We sometimes refer to such probes as “signaling probes” or simply as “On” probes. On probes may be linear (end-labeled linear probes such as those sold as TaqMan® probes for the 5′ exonuclease assay). They may be molecular beacon probes (Tyagi et al. (1996) Nature Biotechnology 14:303-308), including Low-Tm molecular beacons (WO 2006/044994; Sanchez et al. (2004) PNAS (USA) 101: 1933-1938). Molecular beacon probes are single-stranded hairpin-forming oligonucleotides bearing a fluorescent label moiety, typically a fluorophore, on one end, and a non-fluorescent quencher (here one that quenches emission both from the fluorophore and the dsDNA-dye) on the other end. When a molecular beacon probe is in solution, it assumes a closed conformation wherein the quencher interacts with the fluorescent moiety, and the probe is dark. When the probe hybridizes to its target the fluorophore and the quencher moieties are separated and the probe fluoresces in the color of the fluorescent moiety. We sometimes refer to such probes as “Lights-On” or simply “On” probes. An additional type of “On” probe that we have developed is a double-stranded probe having the oligonucleotide structure of yin-yang probes (Li et al. (2002) Nucl. Acids Res. 30, No. 2 e5), wherein the quencher-labeled strand, rather than the fluorescently labeled strand, has a higher Tm.

For use in this invention fluorescent labels of On probes fall, for example, into one of several categories:
a. It may be a “dye-quenching” label, as discussed above, in which event both labels of a hybridized probe will act as quenchers in the dye channel. We sometimes refer to such probes as quenched ResonSense® probes. Emission from the fluorescent label can be acquired by exciting the dye and detecting at or near the maximum emission wavelength of the fluorescent label. Because the fluorescent label is quenched when the probe is free in solution, emission from the fluorescent label can also be acquired by exciting the label directly and detecting at or near its emission maximum. Analysis can include utilization of two fluorescent contours, one from dye emissions and the other from the fluorescent-label emissions.
b. It may be a “dye-coincident” label that is spectrally similar to the dye that is employed such that the stimulating light used to excite the dye also excites the fluorescent moiety, and detection of the dye's emission also detects the fluorescent moiety's emission. For example, the dsDNA-dye may be SYBR® Green and the fluorescent moiety of an On probe may be the fluorophore FAM. SYBR® Green and FAM have nearly identical absorption and emission spectra, but, in contrast to conventional thinking, they can be used together in methods according to this invention to obtain information about the sequences of single-stranded nucleic acid target sequences.
c. It may be a “non-overlapping” label that is spectrally distinct from the dsDNA-dye; that is, it is not excited by the light used to excite the dye, and it does not accept energy from the dye by FRET. An example is the fluorophore Alexa Fluor 790 when used with DAPI dye. Alexa Fluor 790 absorbs at a wavelength longer than DAPI's emission, so excitation of the dye does not excite the label either directly or indirectly. Labels in this category are irrelevant insofar as concerns excitation and detection at wavelengths of the dye, but they can be used to gain information by separate excitation and detection at wavelengths appropriate for the fluorescent label. Analysis can include utilization of the two emission spectra, for example two fluorescent contours, two fluorescent signatures, or both.

Probe sets used in methods of this invention may comprise or include probes that have only one or more dye-quenching labels. If the label or labels are non-fluorescent quenchers, we refer to such probes as “quencher-only” probes or simply as “Off” probes. Quencher-only probes useful in methods of this invention include linear probes and hairpin probes (probes described in WO 2011/0501731, where they are referred to as quencher probes, which is herein incorporated by reference). In some embodiments the non-fluorescent quencher of a quencher-only probe is replaced with a fluorescent moiety that absorbs energy from the dsDNA-dye by FRET. Such a probe is known as a ResonSense® probe. A ResonSense® probe is a single-stranded oligonucleotide labeled with a fluorophore that accepts fluorescence from the dsDNA-dye. Excitation of a sample at the absorption wavelength of the dsDNA-dye results in indirect excitation of the fluorophore, which reemits visible light at a longer wavelength equivalent to the emission spectrum of the fluorophore. The fluorophore is one that is spectrally distinct from the dye. When emission is detected at the wavelength of the dye, the ResonSense® probe acts as a dye-quenching probe, that is, it acts as an Off probe in the dye channel. A ResonSense® probe differs from a quencher-only probe labeled with a non-fluorescent quencher in that, in addition to quenching at the wavelength of the dsDNA-dye, it can also signal at its own emission wavelength when the dye is excited. When a ResonSense® probe is used, analysis generally includes exciting the dye and detecting fluorescence, not only at the emission wavelength of the dsDNA dye, but also separately detecting fluorescence at the emission wavelength of the ResonSense® probe's fluorescent moiety. Off probes other than in-situ probes, and ResonSense® probes typically are simply linear (or random-coil) probes. Structured quencher-only probes are not excluded, however. For example, such a probe could have the hairpin structure of a molecular beacon probe but contain only one or more dye-quenching labels, either fluorescent or non-fluorescent. Or it could have the double-stranded structure of a yin-yang probe (Li et al. (2002) Nucl. Acids Res. 30, No. 2 e5), wherein the probe strand that is complementary to a single-stranded target is labeled only with one or more dye-quenching labels, either fluorescent or, preferably, non-fluorescent quencher, particularly where the quencher-labeled strand has the higher Tm against its target, in this case the single-stranded target sequence being investigated.

Probe sets used in methods of this invention may include On probes, probes that have at least one fluorescent label and at least one non-fluorescent quencher label and that have a structure such that, when not hybridized, the fluorescent label is quenched, but, when hybridized, the fluorescent label is unquenched. Commonly, such On probes are dual-labeled with a fluorophore and a non-fluorescent quencher. When preferred multi-probe sets are hybridized on a target sequence, the fluorescent label of an On probe lies within quenching distance of a non-fluorescent quencher of an Off probe or another On probe. See Example 5, where the fluorophore of On probe 5 is quenched by Off probe 6. In probe sets of this invention, either an On probe or the adjacent probe that quenches it may have the higher Tm. As stated above, certain preferred On probes are single-stranded molecular beacon probes. Another type of useful Off probes is double-stranded and dual-labeled probes that are comprised of a pair of partially complementary oligonucleotides, one of which is labeled with only a non-fluorescent quencher and the other of which is labeled with only a fluorophore (either dye-quenching or non-overlapping), such that when the temperature is lowered in the absence of a target the two strands hybridize to one another, thereby quenching fluorescence of the fluorophore. Probes of this type can be distinct from customary yin-yang probes inasmuch as the non-fluorescent-quencher-labeled strand is the strand that is complementary to the single-stranded target sequence being analyzed. That strand has a higher Tm for the target strand than for the fluorophore-labeled strand of the probe. When the non-fluorescent-quencher-labeled strand binds to its target, it acts like a single-stranded quencher-only probe whose hybridization to the target is detected as decrease in the total dsDNA-dye fluorescence of the system. Detection of fluorescence from now unquenched, single-stranded copies of the fluorophore-labeled strand in a different fluorescent channel is observed as an increase in fluorescence, which serves as a measure of accumulated target strand. The extent of complementarity between the two probe strands is adjustable by altering the composition of either strand, preferably providing that the Tm of the quencher-labeled strand to the target exceeds its Tm to the fluorophore-labeled strand. When that Tm to the target is only a few degrees higher, typically ≦5° C., binding of the quencher-labeled stand to the target will be sequence specific. In contrast, when that Tm to the target is many degrees higher, typically ≧10° C., binding of the quencher strand to the target will be mismatch tolerant. Double-stranded probes in which one strand is quencher-labeled and the other strand is unlabeled are similar except that only one of the strands is labeled. The unlabeled strand does not generate a signal indicative of binding of the quencher-labeled strand to the target strand.

Probe sets useful in methods of this invention can also include one or more unlabeled probes. The gyrase B probe set in Example 3 includes an unlabeled probe that hybridizes to the variable target sequence between two dye-quencher-labeled On probes. In that case the unlabeled probe-target hybrids of two of the variants tested contained two mismatches. Multiple unlabeled probes in a probe set tend to reduce the discriminatory power of the set in the dye channel. In designing a probe set, attention is paid to this tendency, and over-inclusion of unlabeled probes is avoided. Probe sets useful in methods of this invention can include unlabeled in-situ probes.

Design of multi-probe sets for use in methods of this invention is within the ordinary skill of persons familiar with probe design. We describe here several considerations that we take into account. Two primary considerations, as stated above, are the temperature range that is available for detection (we refer to this as the “temperature space”) and the length of of the single-stranded target sequence. Our preferred sets have probes of varying Tm's spread over the target sequence. As to temperature, we generally design a set to have probes with different Tm's against the target sequence, which permits variations in fluorescent contours and fluorescent signatures to occur at multiple temperatures in the detection range. Probes Tm's differing by at least 2° C. will change the fluorescence signature by altering the shape of a valley (or peak). Probes whose Tm's differ by 5° C. or more advantageously lead to separate valleys (or peaks) in the fluorescence signature. However, certain embodiments may include two probes having the same or almost the same Tm, which we refer to as “Tm stacking,” in which case both probes will contribute to a single melting or annealing peak. In designing a probe set for a LATE-PCR amplification reaction, we prefer that the maximum probe Tm against all target sequence variants be below the annealing temperature used in the amplification reaction, which generally is not higher than about 80° C. Design parameters for attaining a desired probe Tm include changing its length, changing its G-C content (adjusting the probe along the target), introducing mismatches, changing the labeling, including non-conventional nucleotides, or introducing structure. Computer programs utilizing the “nearest neighbor” formula are available for use in estimating actual Tm's for set design by calculating probe and primer Tm's against perfectly complementary target sequences and against mismatched target sequences. For Examples in this specification, we have utilized the program Visual OMP (DNA Software, Ann Arbor, Mich., USA), which uses the nearest neighbor method, for calculation of concentration-adjusted Tm's, which we refer to as Tm_{[0]}'s, of primers' or probes' binding sequences against the wild-type or drug-sensitive variant of the target sequence. Particularly for labeled probes, both linear and structured, it will be understood that the Tm so calculated is an approximation. For instance, the data obtained in Example 1 shows that labeling a probe with a Black Hole Quencher 1 reduces the actual Tm of the probe by about 5° C. With in-situ probes, both the initial Tm and the final Tm are taken into account—the initial Tm for construction and the final Tm for detection.

In multiplexing, as shown in Example 3, two probe sets may have Tms in the same range such that fluorescence in the dye channel at a given temperature is influenced by both probe sets. If two or more probes in a probe set or sets have the same or very similar Tm's (i.e. <2° C. difference) their simultaneous hybridization to target is observed by the combined magnitude of the quenched fluorescence at the combined Tm temperature. In contrast, if two or more probes in a set or sets have Tm's that are 2° C. or more separated from one another their simultaneous hybridization to target is observed by their combined quenching over a temperature range that is greater than that of a single probe. For this reason it is generally desirable to design probes in a set or sets whose Tm's differ by at least 2° C., preferably by at least 5° C. and, in certain embodiments by at least 10° C.

As to spacing along the target sequence, adjacent probes in a set may hybridize immediately adjacent to one another. Alternatively, adjacent probes may have overlapping binding sites, provided that the amount of overlap does not preclude either probe from hybridizing (see Example 2, wherein Off probes 2 and 3 overlap by one nucleotide). Alternatively, there may be a gap of a few nucleotides between adjacent probes. We prefer to minimize gaps. We prefer to place a probe having at least a non-fluorescent quencher label complementary to each variable nucleotide in a target sequence. If a target sequence has secondary structure, a hairpin having a certain Tm, we prefer to span the hairpin with two adjacent probes of the equivalent or higher Tm. Probe sets useful in methods according to this invention include dye-quenching probes. If the set includes only one probe, it is a dye-quenching probe, preferably a probe labeled only with one or more non-fluorescent quenchers, most preferably a single, strong non-fluorescent quencher. If the set includes multiple probes, it includes a sufficient number of dye-quenching probes, either quencher-only probes or dual-labeled probes with non-fluorescent quenchers, to be informative regarding the target sequence. As shown in Example 5, that can be determined empirically.

Probes useful in this invention have target-complementary sequences that are generally about 10-40 nucleotides for typical DNA or RNA probes of average binding affinity. The Examples illustrate the use of DNA probes having target-binding sequences 11-30 nucleotides long (low-temperature hairpin probes having the oligonucleotide structure of molecular beacon probes have additional nucleotides that are not complementary to the target but participate in forming a double-stranded stem when the probes are not hybridized to the target sequence). If means are included to increase a probe's binding affinity, the probe can be shorter, as short as seven nucleotides, as persons in the art will appreciate. A label can be attached to a probe at any nucleotide position, including, without limitation, one end of a probe. Columns for synthesis of oligonucleotides may be purchased with a non-fluorescent quencher already attached to what will become the 3′ end of a probe synthesized on the column.

In the case of self-reporting in-situ probes, the length of the probe is determined by several factors: 1) whether or not the 3′ end of the same molecule initially hybridizes to a complementary target sequence; 2) whether the 3′end extends toward the 5′ end; 3) the distance of the initial hybridization sequence from the 5′ end of the molecule. As a result the length of the probe is only predicted in advance. Moreover, the final (extended) length of the probe's stem can be designed to be significantly longer than a conventional probe. A long conventional probe would likely have a Tm above the annealing temperature used for amplification, but a self-reporting in-situ probe having a stem of equivalent length only comes into being after amplification has taken place.

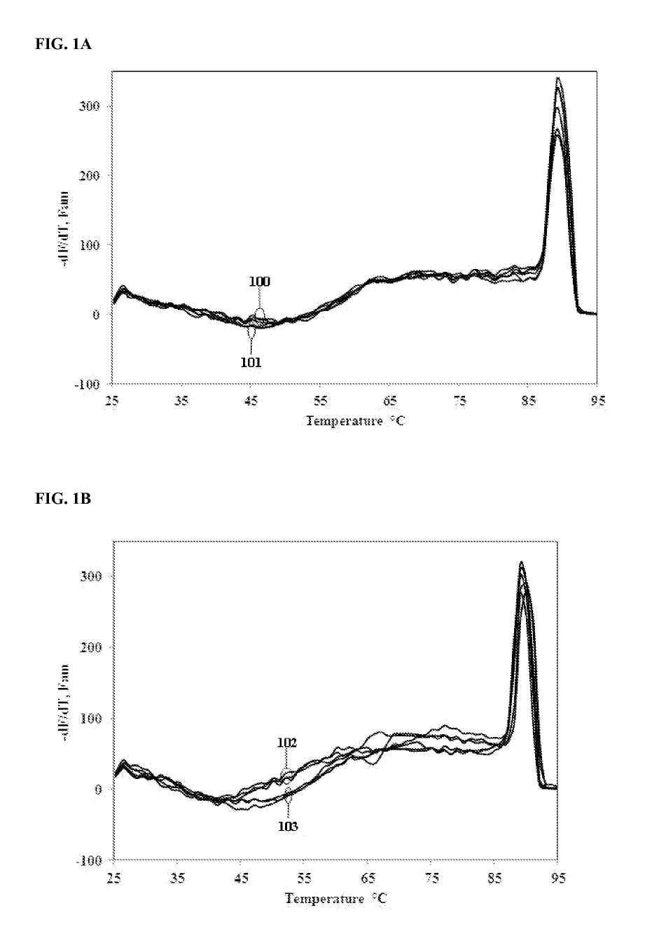

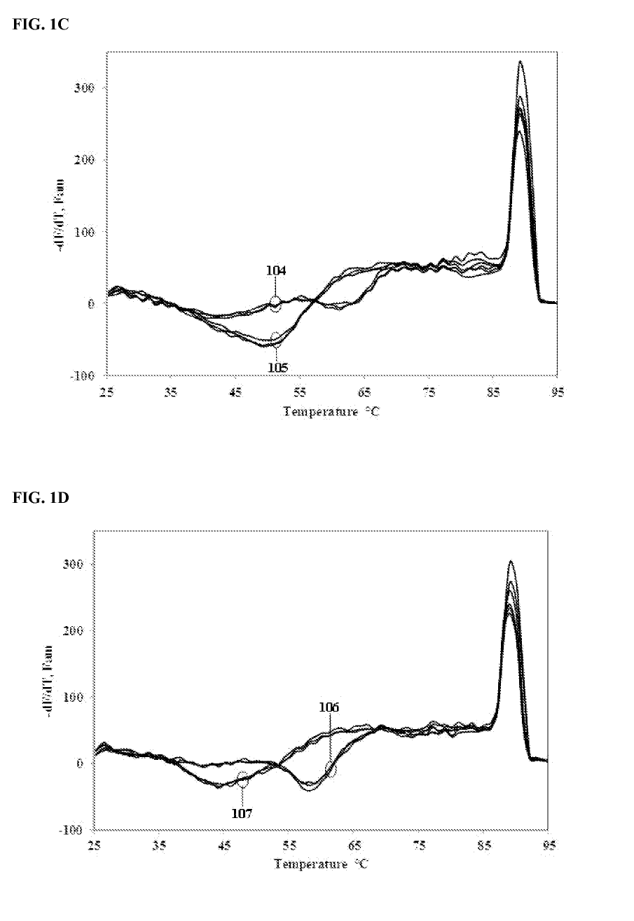

A target sequence to be analyzed by a method of this invention may be, and ordinarily is, a variable sequence. Probes, particularly structured probes, can be constructed so as to hybridize only to perfectly matched variants of target sequences (that is, to be “allele-specific”) at the lowest end of the temperature range utilized for detection, even room temperature. We do not use such probes in single-probe sets in methods of this invention, because, while they differentiate between perfect complementarity and imperfect complementarity, they do not differentiate one imperfect complementarity from another imperfect complementarity. Nor do they generate a fluorescent signature if the target sequence variant is imperfectly complementary to the probes. Allele-specific probes can be included in multi-probe sets for use in methods of this invention, as they will affect the overall signature. It is preferred, however, that most, or in some cases all, probes hybridize to two or more variants of the target sequence at temperatures within the temperature range being utilized. The Tms of such probes will vary with the number and type of mismatches. For instance, the probe in Example 1 was perfectly complementary to a drug-sensitive bacterial strain, but the probe had a single C-to-C mismatch with respect to a drug-resistant strain. As shown in FIG. 1C (fluorescent signatures), the probe hybridized to both variants of the target sequence, but the perfect probe-target hybrid had a negative peak in the SYBR channel at about 62° C., whereas the probe-target hybrid with the mismatch had a negative peak that was shifted to about 50° C. For analyzing a variable sequence to determine which variant is present, a probe set may be designed initially against a wild-type sequence or one bacterial species and then checked to ensure that it produces a different fluorescent signature against a mutant or related bacterial species. In certain embodiments one or more probes can be less than perfectly complementary to every possible target sequence that is anticipated.

In the case of a self-reporting in-situ probe the 3′ end of the variable sequence of interest (sequence 86A in FIG. 8A) lies closer to the 5′ end of the target sequence than it does to the 3′end (sequence 86 in FIG. 8A) to which it is complementary, or partially complementary.

When non-symmetric amplification is employed, we prefer that the probe or probes in the probe set be “low-Tm probes,” that is, probes that do not hybridize to the target sequence during amplification and, therefore, are not cleaved during the reaction. Preferably such probes have Tm's against all suspected or anticipated target sequence variants are at least 5° C. below the Tm of the limiting primer. The extension temperature that is utilized is sufficiently low for extension of the limiting primer but sufficiently high to avoid probe cleavage. When low-Tm probes are employed, detection and analysis of single-strands as a function of temperature can be obtained, not only during post-amplification melting or annealing, but also at intervals during the amplification reaction (for example, all or selected cycles of PCR amplification) by temporarily dropping the temperature below the annealing temperature and then resuming amplification at higher temperatures. To generate a fluorescent contour following amplification, a probe must be available for hybridization during fluorescence acquisition during the detection step. Certain amplification methods, notably the 5′ nuclease (TaqMan®) method, rely on probe cleavage during the amplification reaction, and we employ probe cleavage only for separate analysis of the amount of double-stranded amplicon that is produced during amplification. If only one or more quencher-only probes are used, probe cleavage does not increase background fluorescence in the fluorescent color characteristic of the dsDNA-dye and, therefore, may be used with a probe concentration that is sufficiently high to permit post-amplification hybridization and analysis with uncleaved probes. It is preferred to avoid cleavage of even quencher-only probes, however, so that their concentration does not change in a variable manner prior to end-point analysis. If at least one probe is fluorescently labeled in the color of the dsDNA-dye, its cleavage during amplification produces background fluorescence that will be present during detection and analysis of the single-strand, which is undesirable. In that case probes should be used whose fluorescence does not depend on cleavage, preferably low-Tm probes.

In methods of this invention, a single-stranded target sequence for probing can be provided in any manner that affords sufficient copies for obtaining a melt contour or annealing contour. Many embodiments include nucleic acid amplification utilizing a pair of primers to amplify a target sequence. Amplification may be symmetric, such as symmetric PCR, followed by separation of strands complementary to the single-stranded target sequence to obtain an abundance of single strands containing the target sequence. Alternatively, amplification may be non-symmetric, that is, an amplification method that produces both double-stranded amplicons and an abundance of single-stranded amplicons containing the target sequence. Examples of non-symmetric amplification methods include asymmetric PCR and LATE-PCR. Preferred non-symmetric amplification methods are LATE-PCR methods for starting with DNA or RNA (RT-LATE-PCR). LATE-PCR amplifications and amplification assays are described in, for example, European patent EP 1,468,114 and corresponding U.S. Pat. No. 7,198,897; published European patent application EP 1805199 A2; Sanchez et al. (2004) Proc. Nat. Acad. Sci. (USA) 101: 1933-1938; and Pierce et al. (2005) Proc. Natl. Acad. Sci. (USA) 102: 8609-8614. LATE-PCR is a non-symmetric DNA amplification method employing the polymerase chain reaction (PCR) process utilizing one oligonucleotide primer (the “Excess Primer”) in at least five-fold excess with respect to the other primer (the “Limiting Primer”), which itself is utilized at low concentration, up to 200 nM, so as to be exhausted in roughly sufficient PCR cycles to produce fluorescently detectable double-stranded amplification product (double-stranded amplicon). After the Limiting Primer is exhausted, amplification continues for a desired number of cycles to produce single-stranded amplicon using only the Excess Primer, which we refer to as the Excess Primer strand. LATE-PCR takes into account the concentration-adjusted melting temperature of the Limiting Primer at the start of amplification, Tm_{[0]}^{L}, the concentration-adjusted melting temperature of the Excess Primer at the start of amplification, Tm_{[0]}^{X}, and the melting temperature of the single-stranded amplification product (“amplicon”), Tm_{A}. For LATE-PCR primers, Tm_{[0]} can be determined empirically, as is necessary when non-natural nucleotides are used, or calculated according to the “nearest neighbor” method (Santa Lucia, J. (1998), PNAS (USA) 95: 1460-1465; and Allawi, H. T. and Santa Lucia, J. (1997), Biochem. 36: 10581-10594) using a salt concentration adjustment, which in our amplifications is generally 0.07 M monovalent cation concentration. For LATE-PCR the melting temperature of the amplicon is calculated utilizing the formula: Tm=81.5+0.41 (% G+% C)−500/L+16.6 log [M]/(1+0.7 [M]), where L is the length in nucleotides and [M] is the molar concentration of monovalent cations. Melting temperatures of linear, or random-coil, probes can be calculated as for primers, that is, Tm_{[0]} as described above. Melting temperatures of structured probes, for example molecular beacon probes, can be determined empirically or can be approximated as the Tm_{[0]} of the portion (the loop or the loop plus a portion of the stem) that hybridizes to the amplicon. In a LATE-PCR amplification reaction Tm_{[0]}^{L} is preferably not more than 5° C. below Tm_{[0]}^{X}, more preferably at least as high and even more preferably 3-10° C. higher, and Tm_{A} is preferably not more than 25° C. higher than Tm_{[0]}^{X}, and for some preferred embodiments preferably not more than about 18° C. higher.

Methods of this invention include the step of acquiring dye fluorescence as a function of temperature below the Tm of any double-stranded amplicon. For this purpose the dye is excited at a wavelength at or near its maximum absorption wavelength, and dye emission is detected at a wavelength or wavelengths at or near the dye's maximum emission wavelength, which we refer to as the “dye channel” of an instrument. With numerous instruments, the range for such temperature measurements can be as broad as from about 4° C. to the Tm of double-stranded amplicons, typically about 90-100° C. In large measure the temperature range of each instrument is dependent on the method of cooling. Air-cooled instruments, whether passive or forced convective, typically can only go down to a temperature are about 7-10° C. above ambient temperature, while instruments with artificial cooling, such as a Peltier device (which we refer to as “actively cooled”) can go down to ambient or even below ambient temperatures. Both air-cooled and actively cooled instruments can be used with this invention, although the range of possible temperatures is greater with actively cooled instruments. Instruments can utilize tubes for analysis of the product or chips or microfluidics devices. The optical systems for these instruments can utilize detection in one or channels. In embodiments that include amplification, for example, LATE-PCR, and low-Tm probes, the temperature range over for acquisition of useful information will be capped by Tm's of the probe set at 80-85° C. Certain embodiments include exciting the dsDNA-dye at an appropriate wavelength and detecting emission from the dye within the temperature range at sufficient temperatures needed to generate a fluorescent contour. Quenching of the dsDNA-dye by a non-fluorescent or fluorescent dye-quenching label of a probe bound to a target is, we believe, due to FRET. While the present invention is not limited to any particular mechanism, and an understanding of the mechanism is not necessary to practice the invention, we theorize that the dsDNA-dye in solution first binds to any abundant discrete double-stranded DNA molecules longer than the probes themselves. As the temperature of the reaction is lowered sufficiently for such long molecules to become double-stranded, the dye fluoresces. However, as the temperature is lowered further, a fraction of the already bound dsDNA-dye migrates to the short stretches of double-stranded DNA formed by hybridization of the probe with the dye-quenching label (“On” probe or “Off” probe) to the single-stranded target. When this occurs, the fluorescence of that fraction of dsDNA-dye is quenched by the quencher moiety of the bound probe, thereby reducing the level of total fluorescence in the system below that level that would be observed at the same temperature in the absence of the hybrid formed by the probe and the single-stranded target. In certain embodiments, sufficient information for analysis can be obtained by detecting the dye emission at only two or a few temperatures. If one or more probes in the probe set contain a fluorescent dye-quenching label, the fluorescent dye-quenching label or labels will act as an additional dye-quenching label or additional dye-quenching labels during acquisition in the dye channel. If one or more probes in the probe set contain a fluorescent dye-coincident label or fluorescent dye-coincident labels, the dye-coincident label or labels will generate fluorescence in the dye channel that is independent of the dye's emission. In methods of this invention, acquisition of dye emissions can also include detection at and above the Tm of double-stranded amplicons.

If at least one probe in a probe set includes a spectrally distinct (from the dye) fluorescent label that is indirectly excited by emission from the dsDNA-dye, fluorescence from such label or labels can be similarly but separately acquired at a wavelength or wavelengths at or near that label's maximum emission wavelength, which we refer to as the label's channel, when the dsDNA-dye is excited. Alternatively, fluorescence from such label or labels can be similarly but separately acquired in the label's channel, when the fluorescent label is excited directly. If at least one probe in a probe set includes a spectrally distinct fluorescent label that is not indirectly excited by emission from the dsDNA-dye (a non-overlapping label), fluorescence from such label or labels can be similarly but separately acquired at a wavelength or wavelengths at or near that label's maximum emission wavelength, which we refer to as the label's channel, when the fluorescent label is excited directly. If a probe with a spectrally distinct fluorescent label is a molecular beacon probe, its fluorescence can be separately detected and analyzed to provide additional target sequence-specific information. If a probe with a spectrally distinct fluorescent moiety is a Taqman® probe, its fluorescence can be separately detected and analyzed to provide a measure of the amount of double-stranded DNA amplification that has taken place in a LATE-PCR or asymmetric reaction. If a probe includes a spectrally distinct fluorescent moiety is a double-stranded probe with a fluorophore on the shorter, lower-Tm, strand, its fluorescence can be separately detected and analyzed to provide a measure of the amount of single-stranded DNA amplification in a LATE-PCR or asymmetric amplification reaction.

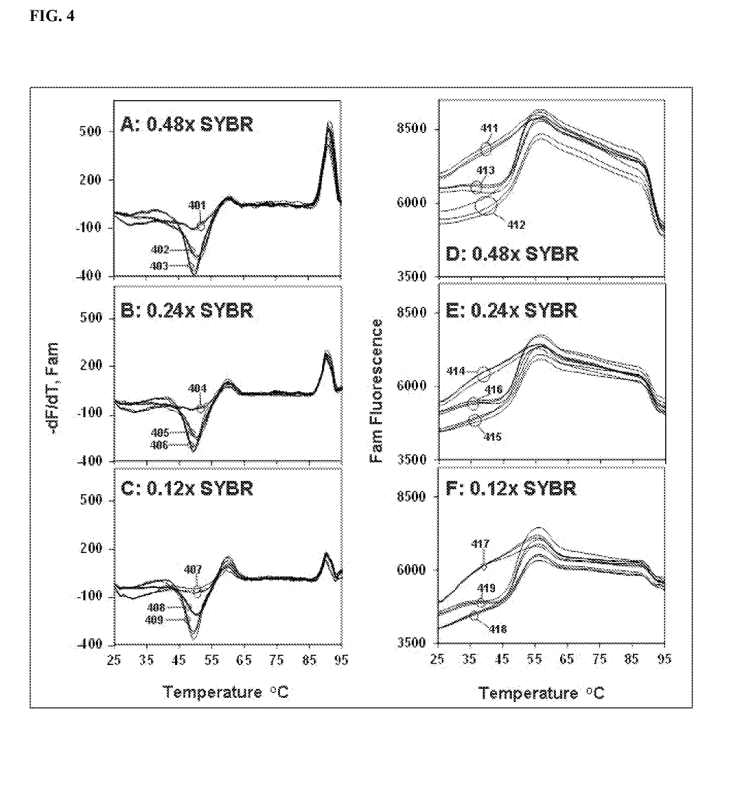

Methods of this invention include comparing emission from the dsDNA-dye as a function of temperature with either a standard or another sample, for example, a sample whose sequence or nature (such as drug susceptibility) is known. A standard is typically a previously obtained result from such a known standard. A comparison may utilize fluorescence contours, such as shown in FIG. 4D. A comparison may utilize fluorescence signatures, such as shown in FIG. 4A. As shown in Example 4 and FIG. 4, panels A-F, one may use a fluorescent signature in addition to or in place of a fluorescent contour. For the purpose of comparing, curves can be normalized. For example, a fluorescent signature can be converted into a normalized fluorescent signature by dividing all values of the fluorescent signature by the lowest valley or the highest positive peak value, if there is a fluorescent label that contributes a fluorescent peak. In certain embodiments, chosen temperatures can be used in place of complete fluorescent contours or fluorescence signatures. Referring to FIG. 1C, one can see that curves 104 and 105 differ from each other at 50° C. and at 62° C. For analytical purposes, one could utilize such fluorescent readings directly, or as a ratio of results at the two temperatures (curve 104 would have a ratio very close to one, whereas curve 105 would have a ratio very different from one). When the data of an anneal fluorescent contour or a melt fluorescent contour is plotted as a first derivative (fluorescent signature) over the full temperature range typically available, about 100° C. to about 25° C., any abundant discrete double-stranded DNA molecules longer than the probes themselves, such as the double-stranded amplicons generated during the exponential phase of a LATE-PCR amplification are detected as a high-Tm positive peak. If fluorescence from the dye is detected at high temperatures approaching 100° C., there will be a melting peak for dissociation of the strands of double-stranded amplicon, if present. Such a peak appears in FIG. 1C, for example, at about 90° C. That melting peak indicates the presence of an abundant discrete double-stranded DNA whose length is greater than that of the probes. Such a peak is typically not sequence-specific and is not detectably altered by minor variations in DNA sequence within the two complementary strands. The fluorescent signature derived by hybridization of the probe to the single-stranded target in the same sample is determined by analysis of the data at a temperature or temperatures below the melt peak of the abundant, discrete double-stranded DNA in the sample. Such a fluorescent signature can be a valley below the background level of the sample in the absence of single-stranded DNA, or it can be a set of valleys, or it can be a set of valleys and peaks below and above the background level of the sample. The precise pattern of fluorescent signature will depend on the sequence of the single-stranded target or targets and the sequence(s) of the probes and the labeling of the probes with dye-quenching labels and fluorophores of the same fluorescent color as the ds-DNA-dye. Moreover, the depth and height of those valleys and peaks will depend on the abundance of the various components of the reaction.

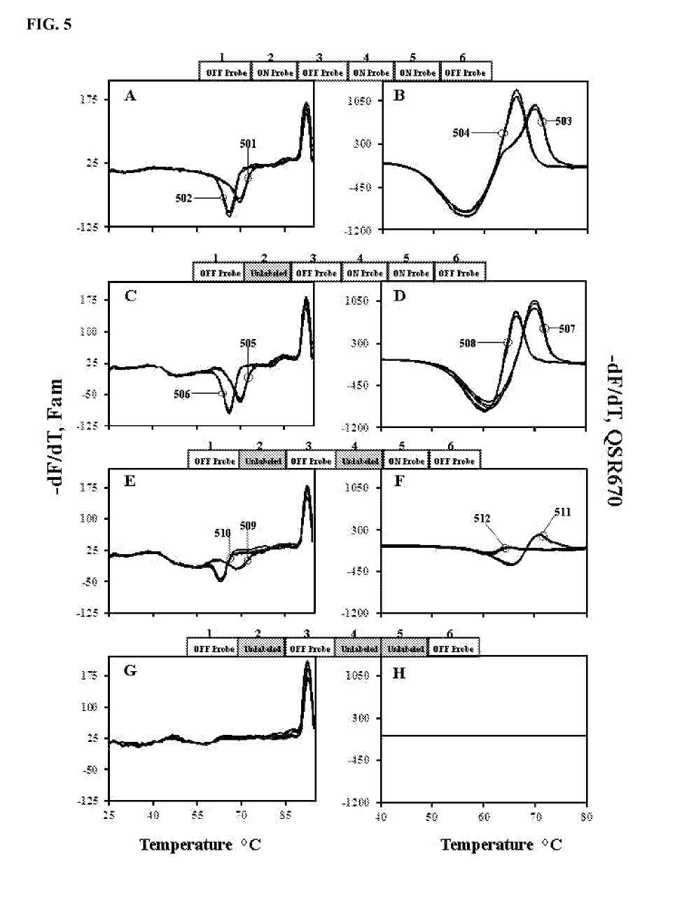

In methods of this invention, the step of comparing can further include comparing emission from a spectrally distinct fluorescent label as a function of temperature with either a standard or another sample in one or more of the various ways used for comparison of dye emissions in order to provide additional information regarding the target sequence or target-sequence variant in a sample. See Example 5, wherein one or more On probes include Quasar 670 fluorophore labels, FIG. 5, panels A, C, and E are fluorescent signatures in the dye channel, and FIG. 5, Panels B, D, and F are fluorescent signatures in the Quasar channel.

Experiments illustrating various aspects of the invention are presented in the Examples. Example 1 describes a method to analyze the sequences of two bacterial strains in a 16 base-pair region of the katG gene utilizing a single quencher-only probe, either a probe with one non-fluorescent quencher moiety or a probe with two non-fluorescent quencher moieties. Example 2 describes a method to analyze the sequences of six bacterial strains in a longer, 101 base-long single-strand of a region of the rpoB gene utilizing a set of multiple (six) singly labeled quencher-only hybridization probes. Example 3 describes a method utilizing SYBR® Green dsDNA-dye in combination with quencher-labeled probes that are also labeled with a FAM fluorophore. Example 5 describes a method utilizing SYBR® Green dsDNA-dye in combination with three quencher-only probes and three quencher-labeled probes that are also labeled with the fluorophore Quasar 670, wherein the six probes hybridize to seven variants of a 101 base long single-strand of a region of the rpoB gene whose initial concentrations prior to amplification are different. The Quasar 670 fluorophore is a red fluorophore that is a FRET partner with SYBR® Green dye. Example 6 describes a method utilizing SYBR® Green dsDNA-dye in combination with a quencher-labeled probe that is also labeled with the fluorophore Quasar 670, which has an emission maximum at 670 nm, wherein the probe by virtue of its hairpin shape serves as a reservoir for the SYBR® Green dsDNA-dye.

Probe sets useful in methods of this invention may include multiple probes of different types for analysis of target sequence variants. For instance, probe sets useful in methods provided herein may include an “On” probe that hybridizes to a target sequence adjacently to another quencher-labeled probe such that the quencher of the latter quenches the fluorescent label of the former when both are hybridized. In Example 3, “On” probe #2 and the “Off” probe in the gyrase B probe set illustrate that possibility. The gyrase B probe set utilized in Example 3 to analyze several species of the genus Mycobacterium includes: a) one quencher-only (“Off”) probe; b) two “On” probes labeled with both a quencher and a FAM; c) one unlabeled probe. Probe sets for multiple unrelated targets may be used together in the same reaction mixture. A method of Example 3 includes a probe set for the gyrase B gene and a probe set for the 16s ribosomal gene in a reaction mixture for amplifying sequences of both genes.

In the Examples, amplification and detection was performed using a Stratagene MX3500P thermal cycler. Detection of SYBR® Green fluorescence was made using the “FAM channel” of the instrument, which detects emission at 516 nm. When a probe or probes containing a FAM label was included, detection included both SYBR® Green fluorescence and FAM fluorescence.

In Example 1, a LATE-PCR amplification was performed using a single pair of primers to amplify a 139 base pair region of the katG gene using either of two strains of Mycobacterium tuberculosis. The amplified single-stranded amplicon generated in the reaction included a 16 base long sequence which is known to contain mutations responsible for drug resistance for isoniazid. Strain 25631 is drug-sensitive and is displayed in FIGS. 1A-1D as curves 100, 102, 104, 106. Strain 8094, which had a single base change (a G to a C) in the 16 base-long sequence is drug-resistant, and is displayed in FIGS. 1A-1D as curves 101, 103, 105, 107. Probes were made with a sequence complementary to the 16 base-long sequence of drug-sensitive strain 25631. One probe was an oligonucleotide without a label. A second probe was the same oligonucleotide with a single Black Hole Quencher 1 (BHQ1) on its 3′ end. A third probe was the same oligonucleotide with a BHQ1 on each end. Four separate amplification reaction mixtures were made with each target strain. Each reaction mixture included 0.24×SYBR® Green which was prepared by dilution from the 10,000× concentration stock provided by the manufacturer. The first mixture did not contain probe. The second mixture contained 500 nM of the unlabeled probe. The third mixture contained 500 nM of the probe with a 3′ BHQ1 modification. The fourth mixture contained 500 nM of the probe with 5′ and 3′ BHQ1's. At the end of amplification, probe-target hybridizations were analyzed as a function of temperature by reading fluorescence from the SYBR® Green at different temperatures (fluorescence acquisition in the FAM channel of the instrument).

FIGS. 1A-1D show the fluorescent signatures that were obtained in Example 1 (first derivative of fluorescence contours as a function of temperature. as temperature was increased). In each case the SYBR® Green dsDNA-dye bound to the abundant double-stranded molecules generated during the exponential phase of the LATE-PCR to generate a melt peak of about 88° C. FIG. 1A presents the results of the fluorescent signatures of both strains tested without a probe. Circle 100 represents strain 25631, and circle 101 represents strain 8094. The two curves are indistinguishable, thereby demonstrating that in the absence of a probe the accumulated single-stranded DNA does not bind SYBR® Green. Only the double-stranded DNA of the amplicon binds SYBR® Green and generates the peak at about 88° C. in the fluorescent signature.

Comparison of curve 102 (FIG. 1B) and curve 100 (FIG. 1A) demonstrates that addition of an unlabeled probe to strain 25631 results in formation of a short region of dsDNA that binds SYBR® Green dye, causing a slight increase in fluorescence over background in the 45-63° C. portion of the fluorescent signature. In contrast, comparison of curves 103 (FIG. 1B) and 101 (FIG. 1A) demonstrates that they are very similar. This indicates that the unlabeled probe alone is unsatisfactory for analyzing the two variants of the variable single-stranded sequence being investigated. FIGS. 1C and 1D illustrate the ability of a method according to this invention that employs only a single quencher-only probe, whether singly labeled with a Black Hole quencher or doubly labeled with two Black Hole quenchers, to distinguish the two variants of the DNA sequence being analyzed through the fluorescent signatures of the dsDNA-dye. FIG. 1C presents the results of the fluorescent signatures of both strains tested with a probe having a single BHQ1 at its 3′ end. Circle 104 represents strain 25631, and circle 105 represents strain 8094. Comparison of curves 104 (FIG. 1C) and 102 (FIG. 1B) shows that the probe with one BHQ1 binds to the single strand of strain 25631 with Tm of about 63° C. and causes a decrease in the level of fluorescence, because the BHQ1 quenches the SYBR® Green that binds to this short region of dsDNA. Comparison of curves 105 (FIG. 1C) and 103 (FIG. 1B) shows that the probe with one BHQ1 binds to the single strand of strain 8094 with Tm of about 53° C. and causes a decrease in the level of fluorescence, because the BHQ1 quenches the SYBR® Green that binds to this short region of dsDNA. Comparison of curves 106 (FIG. 1D) and 102 (FIG. 1B) shows that the probe with two BHQ1 quenchers binds to the single strand of strain 25631 with Tm of about 58° C. and causes a decrease in the level of fluorescence, because the two BHQ1's strongly quench the SYBR® Green that binds to this short region of dsDNA. Comparison of curves 107 (FIG. 1D) and 103 (FIG. 1B) shows that the probe with two BHQ1 quenchers binds to the single strand of strain 8094 with Tm of about 45° C. and causes a decrease in the level of fluorescence, because the two BHQ1's strongly quench the SYBR® Green that binds to this short region of dsDNA. Comparison of curves 106 (FIG. 1D) and 104 (FIG. 1C) shows that the probe with two BHQ1's has a lower effective Tm than the probe with one BHQ1 by 63-58=5° C., and comparison of curves 107 (FIG. 1D) and 105 (FIG. 1C) shows that the probe with two BHQ1's has a lower effective Tm than the probe with one BHQ1 by 53−45=8° C. Thus, the average decrease due to two versus one BHQ1's is about 6.5° C. on average. This decrease in effective Tm is due, we theorize, to the fact that the two BHQ1's on the two ends of the probe stabilize the probe in its unbound state with the result that the effective Tm of the probe to target decreases. Accordingly, in designing a set of probes, one way to separate the Tm's of two probes is by adding a Black Hole quencher to the probe with the lower Tm.

An embodiment that could be an alternative to the method of Example 1 would be to utilize an in-situ Off probe labeled with a Black Hole quencher. In such an embodiment, the excess primer would be labeled with one quencher moiety (here a 5′ BHQ1) and the limiting primer would have a 5′ extension that includes a nucleotide sequence that is not the probe sequence described (SEQ ID No. 5), which is more complementary to drug-sensitive strain 25631 (one mismatch) than to drug-resistant (mutant) strain 8094, but rather is that probe sequence modified to be more complementary to the drug-resistant strain (so that in-situ probes are formed with that strain) and to be very allele-discriminating (for example, as short as practicable, so that in-situ probes are not formed with the drug-sensitive strain).

In Example 2 a LATE-PCR amplification was performed using a single pair of primers to amplify a 150 base pair region of the rpoB gene for each of several strains of Mycobacterium tuberculosis, including drug-sensitive strain 24609 and five different drug-resistant strains, each differing from the drug-sensitive strain by a single base-pair. The single-stranded DNA product (Excess Primer strand) amplified during the linear phase of the LATE-PCR includes a 101 base-long sequence which is known to contain mutations responsible for drug resistance for rifampicin. Six different strains of M. tuberculosis, drug-sensitive strain 24609 and drug resistant strains 18460, 8600, 13554, 14191 and 17718 with each strain tested in triplicate. The products of each of the six closed-tube reactions were analyzed at end-point using SYBR® Green dye and a multi-probe set of six probes that were included in the original amplification reaction mixture. The probes spanned the 101 base pairs of the single-stranded nucleic acid target sequence. All probes were labeled with only a single Black Hole Quencher 2 (BHQ2). At the end of amplification, probe-target hybridizations were analyzed as a function of temperature. As in Example 1, hybridizations were characterized by the use of melt profile analysis in the SYBR® Green channel.

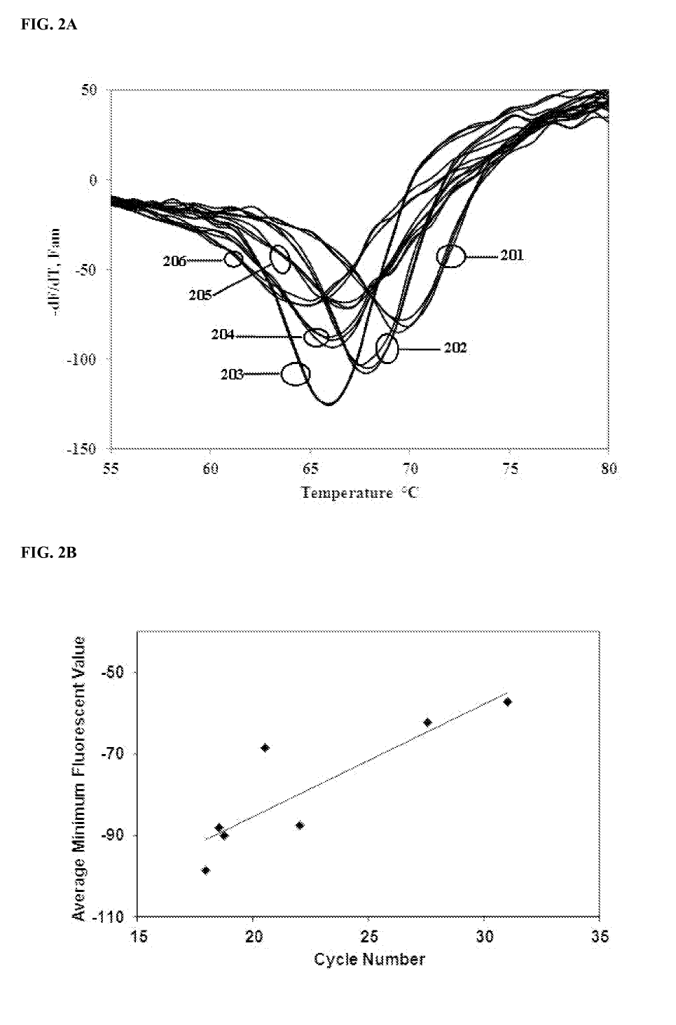

In this example of a method according to this invention the fluorescent signatures, that is, the derivatives of the fluorescent contours obtained by gradually raising the temperature, were compared to one another visually by plotting them on the same graph (FIG. 2). The fluorescent signatures were found to distinguish among all six of the tested strains. Circle 201 represents the drug-sensitive strain 24609. Circle 202 shows resistant strain 18460 (D516V, an aspartic acid located at amino acid position 516 changed to a valine). Circle 203 is the 3 replicates of strain 8600 (L533P, a leucine located at amino acid position 533 changed to a proline). Circle 204 represents drug resistant strain 13554 (H526Y, a histidine located at amino acid position 526 changed to a tyrosine). Circle 205 represents 14191 (H526R, a histidine located at amino acid position 526 changed to a arginine). Circle 206 represents strain 17718 (H526L, a histidine located at amino acid position 526 changed to a leucine). The data show a clear separation of fluorescent signatures for distinguishing each from the other five strains. FIG. 2 illustrates that for each strain the quencher-only (Off) probes in the probe set function together to quench the bound SYBR® Green, but, because of the presence of single-nucleotide variants in the six targets, the composite binding of the six probes varies detectably in a sequence-specific manner. This demonstrates that different drug resistant strains can be distinguished by the method of Example 2.

In Example 3 a multiplex LATE-PCR amplification was used to provide multiple single-stranded target nucleic acid sequences to distinguish M. tuberculosis from other members of the genus Mycobacterium by using a combination of two genes, the 16s ribosomal gene and the gyrase B gene. A target sequence from each gene for one of the target species was amplified in the same tube utilizing two pairs of primers. To investigate and compare differing methods of analysis, three different types of amplification reaction mixtures were tested: a first containing only the 16s probe set, a second containing only the gyrase B probe set, and a third containing both probe sets.

Example 3 demonstrates a method according to this invention that includes the use, in combination with a dsDNA-dye, of unlabeled probes, quencher-only probes, and quencher probes that are labeled with a dye-coincident fluorophore whose excitation and emission spectra are almost the same as the dye's excitation and emission spectra. In Example 3 the dsDNA-dye is SYBR® Green, the most commonly used dye, and the spectrally indistinct fluorophore is FAM. SYBR® Green has an excitation maximum of 497 nm and an emission maximum of 521 nm. FAM is nearly identical, with and excitation maximum of 493 nm and an emission maximum of 525 nm. FAM is included in three probes that we refer to as “On” probes that are quenched when in solution but fluorescent when hybridized. Such “On” probes include a fluorescent moiety, for example a fluorophore or quantum dot, and a non-fluorescent quencher moiety, for example a Black Hole quencher or DABCYL. In this case the set of gyrase B probes included two such “On” probes (gyrase B On Probe #1, gyrase B On Probe #2), one quencher-only probe (gyrase B “Off” probe), and one unlabeled probe that has neither a quencher moiety nor a fluorescent moiety (gyrase B Unlabeled Probe). The set of 16s probes included one “On” probe (16s On Probe) and one quencher-only probe (16s “Off” probe). The “On” probes were all molecular beacons having stems two nucleotides long, labeled on one end with FAM and on the other end with a Black Hole quencher.

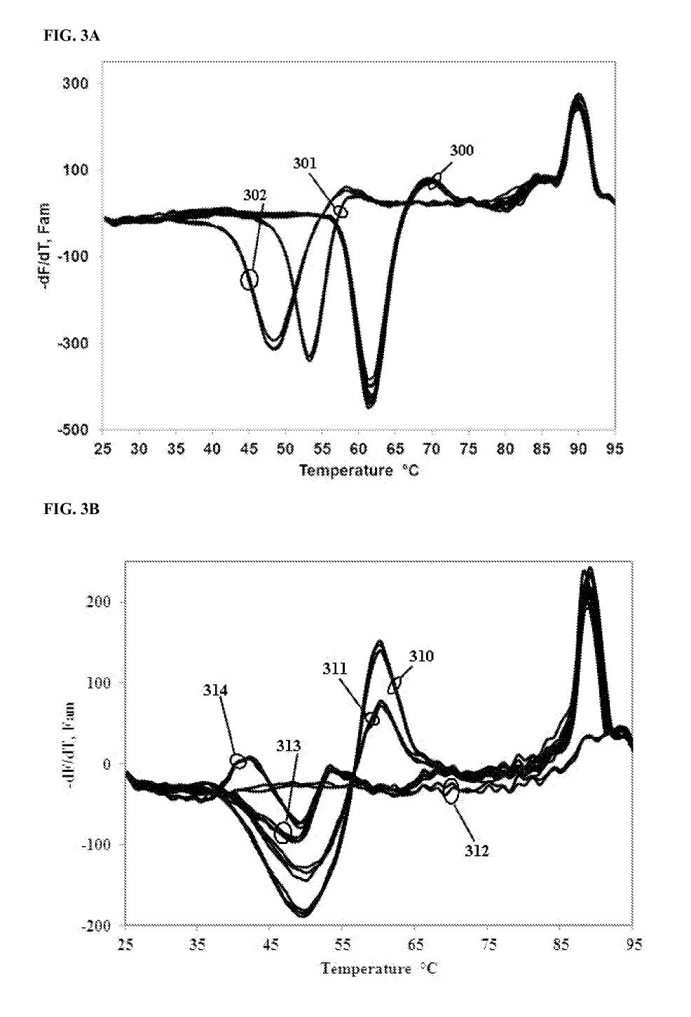

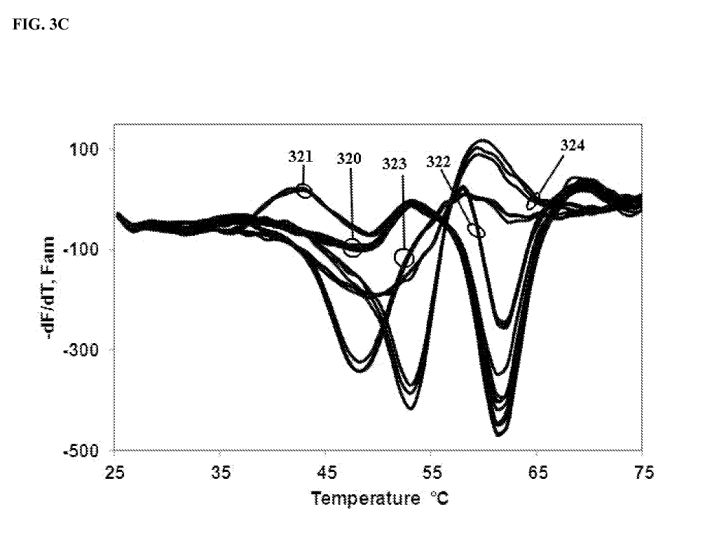

Although both 16s and gyrase B target sequences were amplified in all three reactions described in Example 3, the fluorescent signatures in the dye channel shown in FIG. 3A were obtained by including only SYBR® Green dye and the probe set for the 16s target in the reaction mixture; the fluorescent signatures of FIG. 3B were obtained by including only SYBR® Green dye and the probe set for the gyrase B target in the reaction mixture; and the fluorescent signatures of FIG. 3C were obtained by including SYBR® Green dye and the probe set for the gyrase B target and the probe set for the 16s target in the reaction mixture. Each of these analyses provided different sequence information regarding the target species in the reaction mixture. The reaction mixture of FIG. 3C contained SYBR® Green dye and all six probes, and as a result it provided the most information regarding the target species in the sample. The use of dual-labeled, dye-coincident “On” probes with the SYBR® Green provided distinct and differential fluorescent signatures that characterized members of the Mycobacterium tuberculosis complex (MTBCs) from other members of the genus Mycobacterium, known as NTMs, non-tuberculosis Mycobacterium. FIG. 3A includes melting fluorescent signature 300 for the 16s target sequence for members of the Mycobacterium tuberculosis complex (MTB complex) M. tuberculosis, M. bovis, M. mircoti, and M. africanum, which share a single target sequence. FIG. 3A also includes fluorescent signatures for two NTM's, M. asiaticum (circle 301) and M. avium (circle 302). These fluorescent signatures clearly separate the NTM species from MTB complex. FIG. 3B includes melting fluorescent signatures for M. tuberculosis (circle 310), M. microti and M. africanum (circle 311). M. bovis (circle 312), M. avium (circle 313), and M. asiaticum (circle 314). As can be seen from FIG. 3B, the fluorescent signatures for the gyrase B probes set distinguish members of the MTB complex from one another as well as from each of the NTM species. M. tuberculosis (circle 310) has a sharp peak at 61° C. and a negative peak at 49° C. M. microti and M. africanum, whose gyrase B target sequences are identical, (circle 311) have a peak at 54° C. and minor peak at 49° C. M. bovis (circle 312) has positive peaks at 54° C. and 42° C. with a minor negative peak at 49° C. The NTM species M. avium (circle 313) shows no positive or negative peaks. Example 3 revealed something interesting about the M. asiaticum sample that we obtained. In FIG. 3B, the M. asiaticum (circle 314) sample shows the signature of M. tuberculosis, indicating to us that this sample is a mixture. This was confirmed by sequencing of the 16s and gyrase B sequences from this sample. The 16s sequence data shows that it is M. asiaticum while the gyrase B sequence that of M. tuberculosis. Thus, a mixed sample was analyzed. FIG. 3C includes melting fluorescent signatures for M. microti and M. africanum (common target sequence, circle 320), M. bovis (circle 321), M. tuberculosis (circle 322), M. avium (circle 323), and M. asiaticum (circle 324). As can be seen in FIG. 3C, for the MTB complex members, M. bovis (circle 321) and M. tuberculosis (circle 322) are distinctly different from one another and from M. microti and M. africanum (circle 320). For the NTM species, M. avium (circle 323) is distinct from all other signatures while the mixture sample of M. asiaticum (circle 324) is also unique.

The method of Example 4 is similar to the method of Example 3 regarding dye and probes. However, in this case the primers amplify only the 16s target sequence, and, therefore, only the 16s target sequence produces double-stranded amplicon and single-stranded amplicon. In this Example we varied the dsDNA-dye concentration, and we varied the non-fluorescent quencher of the Off probe (the On probe remained a molecular beacon labeled with a Black Hole quencher and FAM, a dye-coincident label). Fluorescent contours and fluorescent signatures (first derivative) are presented in FIG. 4, panels A-F.

Example 4 demonstrates the effect of concentration of dsDNA-dye, in this case SYBR® Green dye concentration. SYBR® Green at 0.72× inhibited amplification (results not shown). FIG. panels D, E and F show that SYBR® Green dye at 0.48×, 0.24×, and 0.12× did not inhibit amplification. Indeed, the total amount of double-stranded amplicon and single-stranded amplicon was the same in all three sets of reactions. However, as shown by the increase in fluorescence as one moves from about 90° C. to about 85° C. in panels D-F, when the SYBR® Green concentration was decreased from 0.48×, to 0.24×, to 0.12×, the amount of SYBR® Green bound to the double-stranded amplicon decreased. As the temperature decreased further between 85-63° C., the rate of binding of additional SYBR® Green to the double-stranded amplicon also decreased. This indicates that the 0.12× and 0.24×SYBR® Green dye did not saturate the double-stranded amplicon. In contrast, panels D-F show that the amount of SYBR® Green bound to the hybrid of the single-stranded amplicon and the FAM-labeled On probe below about 63° C. was approximately the same in all cases, as was the amount of SYBR® Green bound to the hybrid of the single-stranded amplicon and the three types of OFF probes, at temperatures below about 60° C. This was unexpected. It indicates that in the temperature range of probe-target hybridization, SYBR® Green dye preferentially binds to probe-target hybrids as compared to the double-stranded amplicon. This is consistent with the fact that at end point the concentration of single-stranded amplicon is much higher (10-20 fold) than the concentration of double-stranded amplicon.

Example 4 demonstrates a simple, straightforward empirical approach to optimize the level of dsDNA-dye needed to achieve maximum temperature-dependent subtleties in the resulting melt contours and their first derivatives. In this example 0.12×SYBR® Green was judged to be optimal, because each of the peaks and valleys is most resolved and most reproducible.

Example 4 also illustrates that the magnitude of decrease in SYBR® Green dye signaling that results from hybridization of Off probes to the target sequence. Several alternative non-fluorescent quencher labels were tested for the Off probe, which hybridized adjacent to the On probe. Each variant of the OFF probe had at least one quencher at the 5′ position adjacent to the 3′ FAM of the On probe. One Off probe was labeled with a single 5′ DABCYL, one OFF probe was labeled with both a 5′ DABCYL and a 3′ DABCYL, and one Off probe was labeled with a 5′ BHQ1. As one versed in the art will appreciate, it is also possible to construct Off probes with two BHQ1 quenchers, or with other quenchers, including Black Hole quenchers other than BHQ1. Such alternative quenchers will have different spectra.

FIG. 4 demonstrates that all three variants of the Off probe had sufficient quenching capacity to quiet all or most of the FAM signal from the adjacent On probe. However, as shown by curves 411, 414, 417, the probe with only a single 3′ DABCYL only partially lowered the level of SYBR® Green signaling to background levels over a broad temperature range. In contrast, the probe with two DABCYLs (curves 412, 415, 418) or one BHQ1 (curves 413, 416, 419—) had the capacity to absorb the energy of both the FAM fluorphore and the bound SYBR® Green dye over a relatively small temperature range. The probe with one BHQ1 achieved quenching over a slightly narrower temperature range than the probe with two DABCYLs. Under optimal conditions such as those shown in FIG. 4, panel C, the depth of the “fluorescence valley” in the fluorescent signature becomes deeper in a stepwise manner as the quenching “strength” of the probe progresses: no quencher, one DABCYL, two DABCYLs, one BHQ1. As one versed in the art will appreciate a probe with two BHQ1 absorbs more energy than a probe with one BHQ1 (results not shown) and could have an even deeper “fluorescence valley” than that of the probe with one BHQ1.

FIG. 4 illustrates that two probes may interact when hybridized adjacently on a target sequence. Comparison of circles 402, 405, 408 to circles 403, 406, 409, in FIG. 4, panels A-C, shows that the temperature at which the maximum rate of quenching occurs with a probe having two DABCYLs is consistently a few degrees higher than the maximum rate of quenching for a probe with one BHQ1. This result is consistent with more stable interaction between the 3′ DABCYL and the 5′ FAM of the adjacent On probe, as compared to the interaction between the 3′BHQ1 and the 5′ FAM of the adjacent On probe. The principle of probe/probe interactions can be expanded in many ways, including the following. 1) Adjacent probes can stabilize each other by interaction of either added chemical moieties or single-stranded ends on the adjacent probes. All such “stabilizing” effects of adjacent probes will result in an increase in the effective Tm's of the probes when they are bound to the target strand and melted off by increasing temperature. This increase in effective Tm will contrast to the Tm of each probe as a single probe. 2) Adjacent probes can destabilize each other by interaction of either added chemical moieties or by overlapping of single-stranded ends such that the adjacent ends of the two probes compete for the same target sequence. All such “destabilizing” effects of adjacent probes will result in a decrease in the effective Tm of at least one of the adjacent probes. This decrease in effective Tm will contrast to the Tm of each probe as a single probe. Stabilizing and destabilizing effects can involve adjacent probes that both do not have chemical moieties on their interacting ends, adjacent probes that both do have chemical moieties on their interacting ends, or adjacent probes only where only one of the pair has a chemical moiety on an interacting end. As one versed in the art of probe design will readily appreciate, the impact of both stabilizing and destabilizing probe-probe interactions on a target will have subtle effects on the overall fluorescent profiles of the pair of adjacent probes. For instance, if two probes have similar Tm's and their binding to the target is destabilized by competition of their adjacent ends, the presence of a mutation under one probe will significantly lower its Tm and thereby decrease its competitiveness relative to the other probe. Conversely, if two adjacent probes interact in a manner that stabilizes their hybridization to the target, the presence of one probe will diminish the impact of a mismatch caused by a mutation under the other probe.

An embodiment that could be an alternative to the method of Example 4 would be to utilize an in-situ probe as the Off probe. The in-situ Off probe could be labeled with a Black Hole quencher by using a Black Hole quencher-labeled excess primer, or it could be unlabeled. The limiting primer would have a 5′ extension that includes a nucleotide sequence that is the complement of the probe sequence described (SEQ ID No. 34) modified to be very allele discriminating in favor of the and against the sequence of M. scrofulaceum. The On probe could then be made complementary to M. scrofulaceum. The binding sequence for the On probe would then reside in the loop of the final in-situ probe, lowering its Tm against the M. tuberculosis complex sequence.

Example 5 illustrates the use of a dsDNA-dye (SYBR® Green) and sets of multiple (six) probes that include, in addition to multiple (three) Off probes, additional (three) probes that are either unlabeled or dual-labeled On probes having a dye-quenching fluorophore in an assay to discriminate strains of Mycobacterium tuberculosis that are drug susceptible or drug resistant for the antibiotic rifampicin due to point mutations in the rpoB gene target.

A LATE-PCR amplification was performed using the same single pair of primers used in Example 2 to amplify a 150 base pair region of the rpoB gene for each of two strains of Mycobacterium tuberculosis. The amplification provided a 101 base long single-stranded target (Excess Primer Strand), which includes the RRDR region known to contain mutations responsible for drug resistance for rifampicin. Each single-stranded nucleic acid target sequence was probed using one of four different sets of six probes that were present in the original amplification reaction mixture. Probe-target hybridizations were analyzed as a function of temperature at the end of amplification. In this example, hybridizations were characterized by fluorescent signatures in the SYBR channel (stimulation and detection of SYBR® Green fluorescence) and fluorescent signatures in the Quasar channel (direct stimulation and detection of Quasar 670). Example 2, in which all six probes having the same nucleotide sequences were labeled as Off probes, provided an additional comparison for readings in the SYBR channel. The relevant signatures from Example 2 are in FIG. 2, Circles 201 (drug sensitive) and 203 (mutant train 8600).

Probe Set 1:

All probe sets included the same three Off probes, each labeled with a single Black Hole quencher. The first probe set included additionally three On probes. Each On probe was a molecular beacon probe labeled one end with a Black Hole quencher and on the other end with a Quasar 670 fluorophore—a dye-quenching fluorophore when the dye is SYBR® Green. The alignment of the six probes on the target sequence, including the juxtaposition of labels, is described in Example 5. The alignment is also shown in the legend above FIG. 5, panels A and B. With the probes all hybridized to the target sequence, each of the three fluorophores is adjacent to a Black Hole quencher of a neighboring probe: the 3′ Quasar 670 of On probe 2 lies adjacent to the 5′ BHQ2 of Off probe 1; the 3′ Quasar 670 of On probe 4 lies adjacent to the 5′ BHQ2 of Off probe 3; and the 5′ Quasar 670 of On probe 5 lies adjacent to the 3′ BHQ2 of Off probe 6. Thus, when read in the Quasar channel, the probes performed as an On/Off probe set according to published patent application WO 2011/050173. When read in the SYBR channel, all labels act as quenchers.

Referring to FIG. 5, panels A, C, E, and G, the positive peak at about 93° C. is the melting peak of the double-stranded amplicons. Panel A and panel B show that each fluorescent signature clearly distinguished the two bacterial strains. Comparison of Circles 501 and 502 in the SYBR channel, FIG. 5, panel A, shows that the mutation under On Probe 5 shifts the negative peak (valley) to the left, that is, reduces the temperature of the lowest point of the fluorescent signature from 70° C. to about 66° C. That is very similar to comparison of Circle 201 and Circle 203 in FIG. 2. Comparison of Circles 503 and 504 in the Quasar 670 channel, FIG. 5, panel B, shows that the mutation under On Probe 5 shifts the positive peak to the left, that is, reduces the temperature of the highest point of the fluorescent signature from 70° C. to about 66° C. In both cases the reduction in temperature of the major peak is consistent with the decrease in the Tm of On Probe 5. The shift of the temperature of the negative peak of Circle 502 as compared to Circle 501 is due to On-Probe 5 acting as an Off Probe in the SYBR channel. The temperature of the positive peak of Circle 504 coincides with the temperature of the shoulder in the peak of Circle 503, which is due to On-Probe 2 acting as an On Probe in the Quasar 670 channel.

Probe Set 2:

The second probe set was the same as the first, except for one change: an unlabeled oligonucleotide was substituted for On Probe 2. Panel C and panel D show that each fluorescent signature clearly distinguished the two bacterial strains. Comparison of Circles 505 and 506 in the SYBR channel, FIG. 5, panel C, shows that the mutation under On Probe 5 shifts the negative peak (valley) to the left, from 70° C. to about 66° C. Comparison of Circles 507 and 508 in the Quasar 670 channel, FIG. 5, panel D, shows that the mutation under On Probe 5 shifts the positive peak to the left, from 70° C. to about 66° C. Comparison of Circle 501, panel A, with Circle 505, panel C, shows that substitution of the Unlabeled Probe for On-Probe 2 increases signal curvature between approximately 55° C.-66° C. Similarly, comparison of Circle 503, panel B, with Circle 507, panel D, shows that substitution of the Unlabeled Probe for On-Probe 2 eliminates the shoulder at 66° C. and increases the temperature of the valley below it. Comparison of Circles 507 and 508, FIG. 5, panel D, shows that substitution of the Unlabeled Probe for On-Probe 2 causes the amplitude of the peak in Circle 507 to be higher than the amplitude of the peak in Circle 508. This contrasts with the relative amplitudes of the corresponding peaks in FIG. 5, panel B.

Probe Set 3:

The third probe set was the same as the second except for one change: an unlabeled oligonucleotide was substituted for On Probe 4. This left Probe 5 as the only On probe. Panel E and panel F show that each fluorescent signature clearly distinguished the two bacterial strains. The results in FIG. 5, panel F, reflect the fact that only one Quasar-labeled probe is present in this set of probes. As the temperature drops below 75° C. the positive signal of Probe 5 is quickly extinguished by the nearby quencher on Probe 6. This quenching effect is even more pronounced in Circle 512 as compared to 511, because the mutation under Probe 5 lowers its effective Tm so that it is fluorescence is immediately extinguished by the quencher of Probe 6. FIG. 5, panel E reveals that Probe 5 is functioning as an Off probe in the SYBR channel. The valley of Circle 510 (panel E) is more pronounced than the peak of Circle 512, because when Probe 5 is functioning as an Off probe in the SYBR channel, its quenching capacity is not diminished by the effect that the quencher of Probe 6 has on the Quasar of Probe 5. Nevertheless the Tm of Probe 5 is significantly reduced by the presence of the mutation under Probe 5. This is observed by the shift to the left of the valley of Circle 511 as compare 512.

Probe Set 4:

The fourth probe set was the same as the third except for one change: an unlabeled oligonucleotide was substituted for On Probe 5, the probe under which the mutation lies. This left no On probes. Panel G and panel H show that neither fluorescent signature clearly distinguished the two bacterial strains. Because there are no probes in set 4 that are labeled with Quasar 670, there is no signal in the Quasar channel, FIG. 5, panel H. The results in FIG. 5, panel G also show no valley signals in the SYBR channel, even though there are still three Off Probes labeled with BHQ2. FIG. 2 and FIG. 5, panel A, neither of which has an unlabeled probe in the set of six probes, have a total of six strong BHQ2 quenchers in the set, and they have the deepest valleys. FIG. 5, panel C to panel E to panel G show progressive shallowing of the valleys as the number of strong BHQ2 quenchers in the set goes from five to four to three (unlabeled probes progress from zero to one to two to three), with the signature gone when there are only three BHQ2 quenchers (panel G). We believe that, taken together, these results demonstrate that the Probe Set 4, and hence all probe sets, work as a whole. In other words, the total dsDNA-dye binding (here SYBR® Green binding) to all contiguous probes is balanced by the total number of available quenchers.

Example 6 illustrates the use of SYBR® Green dsDNA dye in PCR amplification carried out in a microfluidics device. The method described there uses an excess of a double-stranded DNA oligonucleotide that serves as reservoir of reagent-bound dye that, because it is bound, is thereby prevented from sticking to the walls of the device and is available to bind to then dissociate and bind to double-stranded amplicons and or probe/target hybrids that result from amplification.

Genetic analysis via the polymerase chain reaction (PCR) in miniaturized microfluidics chip devices is highly desirable, because such devices are very rapid, inexpensive, and are highly flexible in their design. A number of microfluidics devices for nucleic acid amplification reactions, particularly PCR, have been described. Such devices range from stationary chambers to flow-through systems, as well as thermal convection-driven PCR devices, using Taqman probes to detect amplification. The materials used for making these microfluidic chip devices include silicon, glass, plastic and polydimethylsiloxane (PDMS). PDMS for chips has attracted considerable attention for its convenience, low cost and a surface that is inert to most PCR reagents. SYBR® Green, the most commonly used dsDNA-dye, would have an obvious advantage for use in microfluidic devices, because it is both well studied and inexpensive. However, because of the high surface/volume ratio in a microfluidic channel or chamber, an informative signal from SYBR® Green is easily lost by adherence of the dye to surfaces during the filling process and also during the PCR cycling process. Cady et al. have clearly shown that the signal is lost during PCR because of the interaction between PDMS and SYBR® Green I. Cady et al. (2005), Real-time PCR Detection of Listeria Monocytogenes Using an Integrated Microfluidics Platform, Sensors and Actuators B: Chemical, 107(1), 332-341. Gonzalez et al. have shown that SYBR® Green is almost completely adsorbed running through long tubing of perfluoralkoxy (PFA), a polymer which is more inert than PDMS. Gonzalez et al. (2007), Interaction of Quantitative PCR Components with Polymeric Surfaces, Biomed Microdevices, 9(2), 261-266. Different additives such as BSA and surfactants have been used as wall-surface treatments before PCR in order to keep reagents from attaching onto the walls of microfluidic channels.

In Example 6 the problem of SYBR® Green's adherence to surfaces in a microfluidics device, even a device made of PDMS, was overcome by including a double-stranded oligonucleotide in the amplification reaction mixture. We believe that the double-stranded oligonucleotide serves as a reservoir to hold and release SYBR® Green. The oligonucleotide may be two complementary strands or, as we used in Example 6, a single strand that forms a hairpin structure having a double-stranded stem region, for example, a molecular beacon probe or similar structure.

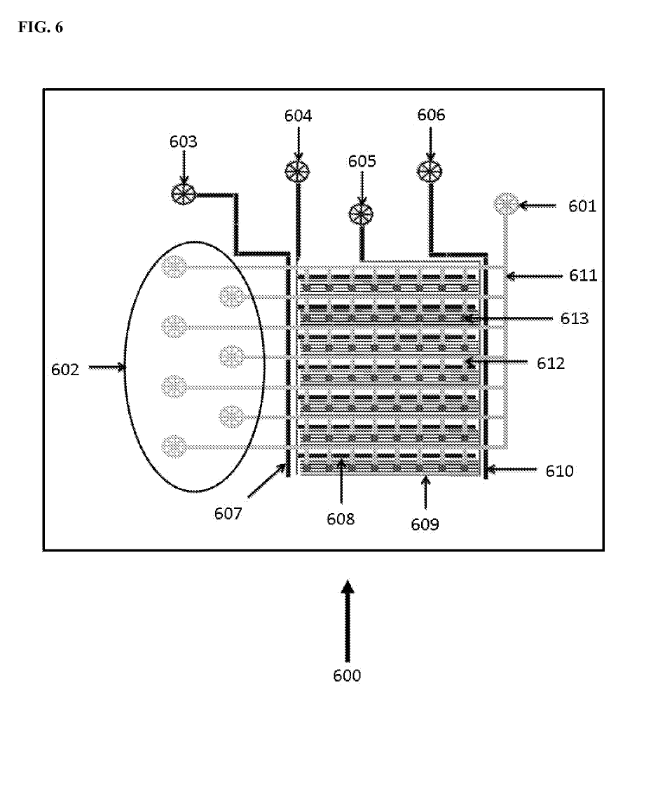

As reported in Example 6, we subjected a series of samples to LATE-PCR amplification. SYBR® Green fluorescence before amplification is shown in FIG. 6, panel C, and SYBR® Green fluorescence after the amplification reaction is shown in FIG. 6, panel D. Panel C shows interaction between SYBR® Green dye and PDMS surfaces of the microfluidics device. Panel D shows that without a double-stranded oligonucleotide in the reaction mixture, the SYBR signal actually decreased during the amplification reaction. In contrast, the sample containing SYBR® Green, molecular beacon probe and target exhibited a marked increase in fluorescence by the end of the amplification reaction, when the reaction mixture contained s small amount of double-stranded amplification product and a larger amount of double-stranded probe-target hybrids. Thus, the double-stranded stem of the hairpin structure of the molecular beacon probe, which exists at room temperature when a device is filled (as was done in the example), serves as a reservoir for SYBR® Green dye. The SYBR® Green bound to the stem is prevented from sticking on the walls of the PDMS chamber and is available to bind to double-strands for detection after amplification. Example 6 demonstrates that under the conditions of that amplification reaction, including particularly 0.96×SYBR® Green and 500 nM molecular beacon, there results a sufficient amount of SYBR® Green in the reaction chamber not bound to the PDMS for SYBR staining of the double-stranded DNA amplicon generated by the reaction.

As one versed in the art will appreciate, other double-stranded molecules, including hairpin oligonucleotides that are not molecular beacons and have stems of various lengths and composition, as well as double strands generated by the hybridization of complementary or partially complementary single-strands, can substitute for the stem of the molecular beacon described here as reservoirs for SYBR® Green dye. In addition, other dyes known to bind to double-stranded DNA can be used instead or in addition to SYBR® Green. The optimal concentrations of said dyes, hairpins, and double-strands can readily be established by experimentation by a person versed in the art.

### EXAMPLES

### Example 1

### Detection of Drug Resistance in the katG Gene for Strains of M. tuberculosis

LATE-PCR amplifications were performed using a single pair of primers to amplify a 139 base pair region of the katG gene for two strains of Mycobacterium tuberculosis. The amplification provided a 16 base-pair region of the gene, which is known to contain mutations responsible for drug resistance for isoniazid, as a single-stranded nucleic acid target sequence. At the end of amplification, probe-target hybridizations were analyzed as a function of temperature. In this example, hybridizations were characterized by the use of melt profile analysis. Reaction components and conditions were as follows:

Limiting Primer:
(SEQ ID No. 1)
5′AGCGCCCACTCGTAGCCGTACAGGATCTCGAGGAAAC
Excess Primer:
(SEQ ID No. 2)
5′TCTTGGGCTGGAAGAGCTCGTATGGCAC
Probe without label
(SEQ ID No. 5)
5′ CTCGATGCTGCTGGTG-C₃
Probe with one quencher moiety
(SEQ ID No. 5)
5′ CTCGATGCTGCTGGTG-BHQ1
Probe with two quencher moieties
(SEQ ID No. 5)
5′BHQ1- CTCGATGCTGCTGGTG-BHQ1

A three carbon linker is denoted with C₃ while a Black Hole Quencher 1 is denoted with BHQ1 (Biosearch Technologies, Novato Calif.). Complementary terminal nucleotides in the probes are underlined.

Target: Strain 25631
(SEQ ID No. 3)
5′GCTTGGGCTGGAAGAGCTCGTATGGCACCGGAACCGGTAAGGACGCGA
TCACCAGCGGCATCGAGGTCGTATGGACGAACACCCCGACGAAATGGGAC
AACAGTTTCCTCGAGATCCTGTACGGCTACGAGTGGGAGCT

The underlined sequence at the 5′ end is the sequence of the excess primer, with the exception that the target has a 5′ terminal G and the excess primer has a 5′ terminal T. The underlined sequence at the 3′ end is the sequence that is complementary to the limiting primer, with the exception that where the fourth nucleotide from the 3′ end of the target sequence is an A, the opposing nucleotide of the limiting primer is G rather than a T. The sequence complementary to the probe sequence is the internal sequence that is italicized and underlined. The probe sequence is perfectly complementary to target strain 25631, with the exception of a T-G mismatch in the middle of the probe sequence.

Target: Strain 8094
(SEQ ID No. 4)
5′GCTTGGGCTGGAAGAGCTCGTATGGCACCGGAACCGGTAAGGACGCGA
TCACCACCGGCATCGAGGTCGTATGGACGAACACCCCGACGAAATGGGAC
AACAGTTTCCTCGAGATCCTGTACGGCTACGAGTGGGAGCT

The underline denotes the location of the single nucleotide change from the drug-sensitive strain. The probe sequence is complementary to target strain 8094 with two micmatches.

LATE PCR amplifications were performed in triplicate in a 25 μl volume consisting of 1×PCR buffer (Invitrogen, Carlsbad, Calif.), 2 mM MgCl₂, 200 nM dNTPs, 50 nM limiting primer and 1000 nM excess primer, 1.25 units of Platinum Taq DNA Polymerase (Invitrogen, Carlsbad, Calif.), 0.24×SYBR® Green (Invitrogen, Carlsbad, Calif.) and one or the other target strain. Strain 25631 was included in an amount of approximately 10,000 genomes equivalents. Strain 8094 was included in an amount of 1,000 genomes equivalents. For each target four separate mixtures were made: the first mixture did not contain the katG probe, the second mixture had 500 nM of the katG probe with no modifications, the third mixture had 500 nM of katG probe with a 3′ BHQ modification, and the fourth mixture had 500 nM of katG probe with 5′ and 3′ BHQ's.

The thermal profile performed on the Stratagene MxPro 3500P for amplification was as follows: 95° C./3 min for 1 cycle, followed by 60 cycles of 98° C./10s-75° C./40s with fluorescent acquisition at each cycle. This was followed by one cycle of 10 min at 75° C. and 10 min at 25° C. This was followed by a melt with fluorescence acquisition in the FAM channel (excitation, 492 nm, emission, 516 nm) at each degree starting at 25° C. with 1° C. increments at 30s intervals to 97° C. Probe-target hybridizations were analyzed by melt curve analysis using the first derivative.

FIGS. 1A-D shows the results of the fluorescent signatures (derivative of melting curves) for each mixture set. FIG. 1A presents the results of the melt curve analysis of both strains tested without a probe. Circle 100 identifies the replicates containing strain 25631, and circle 101 identifies the replicates containing strain 8094. FIG. 1B shows both strains tested with a probe that does not have a quencher moiety. Circle 102 identifies the replicates containing strain 25631 and circle 103 identifies the replicates containing strain 8094. FIG. 1C shows both strains tested with a probe with a single quencher. Circle 104 identifies the replicates containing strain 25631, and circle 105 identifies the replicates containing strain 8094. FIG. 1D shows both strains tested with a probe with two quencher moieties. Circle 106 identifies the replicates containing strain 25631, and circle 107 identifies the replicates containing strain 8094.

The Excess Primer contains a deliberate mismatch at the 5′ end (a “T” rather than the “G” in each of the targets) to reduce potential mispriming during the linear phase of LATE-PCR amplification.

### Example 2

### Detection of Drug Resistance in the rpoB Gene for Strains of M. tuberculosis

LATE-PCR amplifications were performed using a single pair of primers to amplify a 150 base pair region of the rpoB gene for each of several strains of Mycobacterium tuberculosis. The amplification provided a 101 base-pair region of the gene, which is known to contain mutations responsible for drug resistance for rifampicin, as a single-stranded nucleic acid target sequence (the Excess Primer strand of each LATE-PCR amplification). Following amplification, each single-stranded nucleic acid target sequence was probed using six separate probes that were included in the original amplification reaction mixture.

The probes spanned the 101 base pairs of the single-stranded nucleic acid target sequence. All probes were labeled with BHQ2 only and no fluorophore. In this example the fluorescent signatures are all distinct from one another and differ with respect to the drug-sensitive strain. At the end of amplification, probe-target hybridizations were analyzed as a function of temperature. In this example, hybridizations were characterized by the use of melt profile analysis. Reaction components and conditions were as follows:

Limiting Primer:
(SEQ ID No. 6)
5′-CTCCAGCCAGGCACGCTCACGTGACAGACCG
Excess Primer:
(SEQ ID No. 7)
5′-CCGGTGGTCGCCGCGATCAAGGAG
Probe 1:
(SEQ ID No. 14)
5′- BHQ2-CTGGTTGGTGCAGAAG-C₃
Probe 2:
(SEQ ID No. 15)
5′- BHQ2-TCAGGTCCATGAATTGGCTCAGA- C₃
Probe 3:
(SEQ ID No. 16)
5′- BHQ2-CAGCGGGTTGTT-C₃
Probe 4:
(SEQ ID No. 17)
5′-BHQ2-ATGCGCTTGTGGATCAACCCCGAT- C₃
Probe 5:
(SEQ ID No. 18)
5′-AAGCCCCAGCGCCGACAGTCGTT-BHQ2
Probe 6:
(SEQ ID No. 19)
5′-ACAGACCGCCGG-BHQ2

A three-carbon linker, which blocks extension of a probe, is denoted with C₃ while a Black Hole Quencher 2 is denoted with BHQ2 (Biosearch Technologies, Novato Calif.). Complementary terminal nucleotides in the probes are underlined.

```
Target: Strain 24609
                                                            Probe 1 -Q
5′CCGGTGGTCGCCGCGATCAAGGAGTTCTTCGGCACCAGCCAG
                                            Probe 3 -Q
CTGAGCCAATTCATGGACCAGAACAACCCGCTGTCGGGGTTGACCCACAA
GCGC
A Probe 2 -Q T Probe 4 TAQ
QTT Probe 5 AA
(SEQ ID No. 8)
CGACTGTCGGCGCTGGGGCCCGGCGGTCTGTCACGTGAGCGTGCCTGGCT
GGAG
                                      Q- Probe 6
```

The location of primers and probes is shown relative to strain 24609. The underlined sequence at the 5′ end is the sequence of the excess primer. The underlined sequence at the 3′ end is the sequence that is complementary to the limiting primer. The binding sites of probes 1-6 are indicated, with “-Q” indicating the probe end having the BHQ2 quencher. Non-complementary terminal nucleotides are identified (for example, “TAQ” at the 3′ end of Probe 4 denotes a non-complementary T and a non-complementary A. Probes in this set hybridize immediately adjacently to one another. Probe 6 overlaps the excess primer.

Target: Strain 18460
(SEQ ID No. 9)
5′CCGGTGGTCGCCGCGATCAAGGAGTTCTTCGGCACCAGCCAGCTGAGC
CAATTCATGGTCCAGAACAACCCGCTGTCGGGGTTGACCCACAAGCGCCG
ACTGTCGGCGCTGGGGCCCGGCGGTCTGTCACGTGAGCGTGCCTGGCTGG
AG
Target: Strain 8600
(SEQ ID No. 10)
5′CCGGTGGTCGCCGCGATCAAGGAGTTCTTCGGCACCAGCCAGCTGAGC
CAATTCATGGACCAGAACAACCCGCTGTCGGGGTTGACCCACAAGCGCCG
ACTGTCGGCGCCGGGGCCCGGCGGTCTGTCACGTGAGCGTGCCTGGCTGG
AG
Target: Strain 14191
(SEQ ID No. 11)
5′CCGGTGGTCGCCGCGATCAAGGAGTTCTTCGGCACCAGCCAGCTGAGC
CAATTCATGGACCAGAACAACCCGCTGTCGGGGTTGACCCGCAAGCGCCG
ACTGTCGGCGCTGGGGCCCGGCGGTCTGTCACGTGAGCGTGCCTGGCTGG
AG
Target: Strain 17718
(SEQ ID No. 12)
5′CCGGTGGTCGCCGCGATCAAGGAGTTCTTCGGCACCAGCCAGCTGAGC
CAATTCATGGACCAGAACAACCCGCTGTCGGGGTTGACCCTCAAGCGCCG
ACTGTCGGCGCTGGGGCCCGGCGGTCTGTCACGTGAGCGTGCCTGGCTGG
AG
Target: Strain 13554
(SEQ ID No. 13)
5′CCGGTGGTCGCCGCGATCAAGGAGTTCTTCGGCACCAGCCAGCTGAGC
CAATTCATGGACCAGAACAACCCGCTGTCGGGGTTGACCTACAAGCGCCG
ACTGTCGGCGCTGGGGCCCGGCGGTCTGTCACGTGAGCGTGCCTGGCTGG
AG

The underline in the sequence of each of strains other than strain 24609 denotes the location of the nucleotide change from the drug-sensitive strain 24609.

LATE PCR amplifications were carried out in a 25 μl volume consisting of 1×PCR buffer (Invitrogen, Carlsbad, Calif.), 2 mM MgCl2, 200 nM dNTPs, 50 nM Limiting Primer, 1000 nM Excess Primer, 1.5 units of Platinum Taq DNA Polymerase (Invitrogen, Carlsbad, Calif.), 0.24×SYBR® Green (Invitrogen, Carlsbad, Calif.), 500 nM of all probes and 10,000 genomes of human genomic DNA (Promega, Madison. Wis.). For each strain tested approximately 10,000 genomes equivalents were used. Amplification reactions for each strain were run in triplicate.

The thermal profile performed on the Stratagene MxPro 3005P for amplification was as follows: 95° C./3 min for 1 cycle, followed by 60 cycles of 98° C./10s-75° C./40s with fluorescent acquisition in the FAM channel at each cycle. This was followed by one cycle of 10 min at 75° C. and 10 min at 25° C. This was followed by a melt with fluorescent acquisition at each degree starting at 25° C. with 1° C. increments at 30s intervals to 97° C. Analysis of the probe target hybridizations following amplification was by melt curve analysis using the first derivative of FAM fluorescence intensity for temperatures between 25° C. to 97° C.

FIG. 2 presents the fluorescent signatures from the samples in the temperature range of 55° C. to 80° C. Circle 201 identifies the replicates containing drug-sensitive strain 24609. Circle 202 identifies the replicates containing resistant strain 18460 (D516V, an aspartic acid located at amino acid position 516 changed to a valine). Circle 203 identifies the three replicates containing strain 8600(L533P, a leucine located at amino acid position 533 changed to a proline). Circle 204 identifies the replicates containing drug-resistant strain 13554(H526Y, a histidine located at amino acid position 526 changed to a tyrosine). Circle 205 identifies the replicates containing resistant strain 14191(H526R, a histidine located at amino acid position 526 changed to a arginine). Circle 206 identifies the replicates containing resistant strain 17718(H526L, a histidine located at amino acid position 526 changed to a leucine). The data show a clear separation of fluorescent signatures for all six strains.

### Example 3

### Species Differentiation Among Members of the Genus Mycobacterium

A multiplex LATE-PCR assay was used to provide multiple single-stranded target nucleic acids to identify M. tuberculosis from other members of the genus Mycobacterium by using a combination of two genes, the 16s ribosomal and gyrase B genes. The use of dual labeled probes with the SYBR® Green provided distinct and differential fluorescent signatures that characterized members of the Mycobacterium tuberculosis complex from other members of Mycobacterium. In this example, hybridizations were characterized by the use of melt profile analysis. Reaction components and conditions were as follows:

The primer and probe sequences for gyrase B are:

Limiting Primer:
(SEQ ID No. 20)
5′-GTCAGCGAACCGCAGTTCGAGGGCCAGACCAAGACC
Excess Primer:
(SEQ ID No. 21)
5′-ATACGGGCTTGCGCCGAGGACAC
gyrase B On Probe #1:
(SEQ ID No. 22)
5′- FAM-CGTGTAATGAATAGCTGCG-BHQ1
gyrase B On Probe #2:
(SEQ ID No. 23)
5′-BHQ1-AGGACGCGAAAGTCGTTGCT-C₃- FAM
gyrase B Off Probe:
(SEQ ID No. 24)
5′-BHQ1-TGAACAAGGCT-C₃
gyrase B unlabeled Probe:
(SEQ ID No. 25)
5′-CCACTGGTTTGAAGCCAACCCCA-C₃

A three-carbon linker is denoted with C₃ while a Black Hole Quencher 1 is denoted with BHQ1 (Biosearch Technologies, Novato Calif.). Complementary terminal nucleotides in the On probes are underlined.
The gyrB target sequences are:

```
M. tuberculosis
5′GTCAGCGAACCGCAGTTCGAGGGCCAGACCAAGACCAAGTTGGGCAAC
AC
                                                F-CG On #1 CG-Q
CGAGGTCAAATCGTTTGTGCAGAAGGTCTGTAACGAACAGCTGAC
                                            Q-AG On #2 CT-F
CCACTGGTTTGAAGCCAACCCCACCGACGCGAAAGTCGTTGTGAACAAGG
CT
        Unlabeled Q- Off
(SEQ ID No. 26)
GTGTCCTCGGCGCAAGCCCGTAT
```

The location of primers and probes is shown relative to the M. tuberculosis sequence. The underlined sequence at the 5′ end is the sequence of the limiting primer. The underlined sequence at the 3′ end is the sequence that is complementary to the excess primer. The binding sequences of probes are indicated, with “F: indicating the probe end having the FAM fluorophore. “-Q” indicating the probe end having the BHQ1 quencher, and non-complementary terminal nucleotides identified (for example, “FCG” at the 5′ end of the On#1 sequence denotes a non-complementary C and a non-complementary G. We note that probes in the set hybridize immediately adjacently to one another except that there is a two-nucleotide (CC) gap between the sequence of the unlabeled probe and the binding sequence of On Probe 2. For M. asiaticum (see below), which has a C-to-G difference at this point, the gap is only a single nucleotide.

M. bovis
(SEQ ID No. 27)
5′GTCAGCGAACCGCAGTTCGAGGGCCAGACCAAGACCAAGTTGGGCAAC
ACCGAGGTCAAATCGTTTGTGCAGAAGGTCTGTAATGAACAGCTGACCCA
CTGGTTTGAAGCCAACCCCACCGACTCGAAAGTCGTTGTGAACAAGGCTG
TGTCCTCGGCGCAAGCCCGTAT
M. microti
(SEQ ID No. 28)
5′GTCAGCGAACCGCAGTTCGAGGGCCAGACCAAGACCAAGTTGGGCAAC
ACCGAGGTCAAATCGTTTGTGCAGAAGGTCTGTAACGAACAGCTGACCCA
CTGGTTTGAAGCCAACCCCACCGACTCGAAAGTCGTTGTGAACAAGGCTG
TGTCCTCGGCGCAAGCCCGTAT
M. africanum (same sequence as M. microti)
M. avium
(SEQ ID No. 29)
5′GTGAGCGAACCGCAGTTCGAGGGCCAGACCAAGACCAAACTGGGCAAC
ACCGAGGTGAAGTCGTTCGTGCAGAAGGTGTGCAACGAACAGCTCACCCA
CTGGTTCGAAGCCAACCCCGCAGACGCCAAAGTCATTGTCAACAAGGCGG
TTTCGTCAGCGCAGGCGCGCAT
M. asiaticum
(SEQ ID No. 30)
5′GTCGCCGAACCCCAGTTCGAGGGCCAGACAAAGACCAAGCTGGGCAAC
ACCGAGGTCAAGTCGTTCGTGCAGAAGGTGTGCAACGAACAGCTCACCCA
CTGGTTCGAGGCCAATCCGTCGGAAGCCAAAACCGTTGTCAACAAGGCGG
TTTCGTCCGCACAGGCCCGGAT

The underlines in the target sequence of each species other than M. tuberculosis denotes the location of nucleotide changes from the M. tuberculosis sequence.
The primer and probe sequences for 16s are:

Limiting Primer:
(SEQ ID No. 31)
5′-ACACCCTCTCAGGCCGGCTACCCGTCG
Excess Primer:
(SEQ ID No. 32)
5′-GAGTGGCGAACGGGTGAGTAACACG
16s On Probe:
(SEQ ID No. 33)
5′-BHQ1-TTGGCTCATCCCACACCGCTAAAGTGCTTTAA-FAM
16s Off Probe:
(SEQ ID No. 34)
5′-BHQ1-CCACCACAAGATATGCGTCTCGTGTTCCTAT-C3

A three-carbon linker is denoted with C₃ while a Black Hole Quencher 1 is denoted with BHQ1 (Biosearch Technologies, Novato Calif.). Complementary terminal nucleotides in the On probe are underlined.

### 16s Target Sequences:

Members of Mycobacterium tuberculosis complex, including M. tuberculosis, M. africanum, M. bovis and M. microti

5′GAGTGGCGAACGGGTGAGTAACACGTGGGTGATCTGCCCTGCACTTCG
GGATAAGCCTGGGAAACTGGGTCTAATACCGG
Off Probe -Q
ATAGGACCACGGGATGCATGTCTTGTGGTGG
F-AA On Probe TT-Q
AAAGCGCTTTAGCGGTGTGGGATGAGCCCGCGGCCTATCAGCTTGTTG
(SEQ ID No. 35)
GTGGGGTGACGGCCTACCAAGGCGACGACGGGTAGCCGGCCTGAGAGGGT
GT

The location of primers and probes is shown relative to the MTB-complex sequence. The underlined sequence at the 5′ end is the sequence of the excess primer. The underlined sequence at the 3′ end is the sequence that is complementary to the limiting primer. The binding sites of the probes are indicated, with “F-” indicating the probe end having the FAM fluorophore and “-Q” indicating the probe end having the BHQ1 quencher. Non-complementary terminal nucleotides are identified (for example, “F-AA” at the 5′ end of the On Probe sequence denotes two non-complementary A's. The two probes in the set hybridize immediately adjacently to one another, that is, there is neither an overlap nor a gap. Comparing the sequence of the On probe to its MTB-complex binding site, the sixth nucleotide from the 5′ end of the binding site (a G) is a mismatch, and the next nucleotide (a C) has no counterpart in the probe sequence (the probe sequence has a deletion).

### Non-Tuberculosis-Complex Mycobacteria:

M. asiaticum
(SEQ ID No. 36)
5′GAGTGGCGAACGGGTGAGTAACACGTGGGTGATCTGCCCTGCACTTCG
GGATAAGCCTGGGAAACTGGGTCTAATACCGGATAGGACCACGGGATGCA
TGTCCTGTGGTGGAAAGCTTTTGCGGTGTGGGATGGGCCCGCGGCCTATC
AGCTTGTTGGTGGGGTGACGGCCTACCAAGGCGACGACGGGTAGCCGGCC
TGAGAGGGTGT
M. avium
(SEQ ID No. 37)
5′GAGTGGCGAACGGGTGAGTAACACGTGGGCAATCTGCCCTGCACTTCG
GGATAAGCCTGGGAAACTGGGTCTAATACCGGATAGGACCTCAAGACGCA
TGTCTTCTGGTGGAAAGCTTTTGCGGTGTGGGATGGGCCCGCGGCCTATC
AGCTTGTTGGTGGGGTGACGGCCTACCAAGGCGACGACGGGTAGCCGGCC
TGAGAGGGTGT

The underlines in the target sequence of each non-tuberculosis-complex species denotes the location of nucleotide changes from the Mycobacterium tuberculosis complex sequence.

LATE-PCR amplifications were performed in triplicate in a 25 μl volume consisting of i×PCR buffer (Invitrogen, Carlsbad, Calif.), 0.24×SYBR® Green, 2 mM MgCl₂, 300 nM dNTPs, 50 nM limiting primers, 1000 nM of excess primers, 1.5 units of Platinum Taq DNA Polymerase (Invitrogen, Carlsbad, Calif.). Three separate mixtures were made; the first had only the gyrase B probe set: 200 nM of gyrase B On Probe #1, 200 nM of gyrase B On Probe #2, 500 nM of gyrase B Off Probe and 1 uM of gyrase B Unlabeled Probe. The second mixture had only the 16s probe set: 200 nM of the 16s On Probe and 500 nM of 16s Off Probe. The third mixture included both probe sets (six probes).

The thermal profile performed on the Stratagene MxPro 3005P for amplification was as follows: 98° C./3 min for 1 cycle, followed by 98° C./10s-75° C./40s for 60 cycles, followed by 10 min at 75° C., followed by 10 min at 25° C. with a melt starting at 25° C. with 1° C. increments at 30s intervals to 97° C. with fluorescent acquisition in the FAM channel (excitation, 492 nm, emission, 516 nm) at each degree. Probe-target hybridizations were analyzed by the melt curve analysis using the first derivative for the temperatures between 25° C. to 95° C.

FIG. 3A-C shows the results of the melt derivatives for the mixtures. FIG. 3A presents the fluorescent signatures for mixtures that included the 16s probes. Circle 300 identifies the replicates for the MTB-complex target samples. In this assay M. tuberculosis, M. bovis, M. mircoti, and M. africanum have identical fluorescent signatures. Circle 301 identifies the replicates for M. asiaticum. Circle 302 identifies the replicates for M. avium. FIG. 3B presents the fluorescent signatures for mixtures that contained the gyrase B probes. Circle 310 identifies the replicates for M. tuberculosis. Circle 311 identifies the replicates for M. microti and M. africanum. Circle 312 identifies the replicates for M. bovis. Circle 313 identifies the replicates for M. avium. Circle 314 identifies the replicates for M. asiaticum. FIG. 3C presents the fluorescent signatures for mixtures that included all probes. Circle 320 identifies the replicates for M. microti and M. africanum, which share the same fluorescent signature. Circle 321 identifies the replicates for M. bovis. Circle 322 identifies the replicates for M. tuberculosis. Circle 323 identifies the replicates for M. avium. Circle 324 identifies the replicates for M. asiaticum.

### Example 4

### The Effect of SYBR Concentration and Quencher Type and Number

A LATE-PCR assay was used to provide single-stranded target nucleic acids. The amplification reaction mixture contained two genes, the 16s ribosomal and the gyrase B genes of M. simiae, but the primers for gyrase B did not amplify this species, so only single-stranded amplification product for the 16s gene resulted. The use of dual-labeled On probes with the SYBR® Green provided distinct and differential fluorescent signatures based on the combinations and the type of quenchers used. In this example, hybridizations were characterized by the use of melt profile analysis. Reaction components and conditions were as follows:

The gyrase B Limiting Primer, Excess Primer, and Probes were the same as in Example 3.
The gyrase B target sequence for M. simiae is not available from genbank.
The primer and probe sequences for 16s are:
Limiting Primer: same as in Example 3
Excess Primer: same as in Example 3
16s On Probe: (same as in Example 3)

(SEQ ID No. 33)
5′-BHQ1-TTGGCTCATCCCACACCGCTAAAGTGCTTTAA-FAM

16s Off Probe with Black Hole Quencher 1: (same as in Example 3)

(SEQ ID No. 34)
5′-BHQ1-CCACCACAAGATATGCGTCTCGTGTTCCTAT-C3

16s Off Probe with one DABCYL (D):

(SEQ ID No. 34)
5′-DABCYL-CCACCACAAGATATGCGTCTCGTGTTCCTAT-C3

16s Off Probe with two DABCYLs (DD):

(SEQ ID No. 34)
5′-DABCYL- CCACCACAAGATATGCGTCTCGTGTTCCTAT-DABCYL

A three carbon linker is denoted with C₃ while a Black Hole Quencher 1 is denoted with BHQ1 (Biosearch Technologies, Novato Calif.). Complementary terminal nucleotides in the On probe are underlined.

### 16s Target Sequence:

M. simiae
(SEQ ID No. 38)
5′CTCGAGTGGCGAACGGGTGAGTAACACGTGGGTAATCTGCCCTGCACT
TCGGGATAAGCCTGGGAAACTGGGTCTAATACCGG
Off Probe Q
ATAGGACCACTTGGCGCATGCCTTGTGGTGG
F-AA On Probe TT-Q
AAAGCTTTTGCGGTGTGGGATGGGCCCGCGGCCTATCAGCTTGTTG
GTGGGGTGACGGCCTACCAAG GCGACGACGGGTAGCCGCCCTGAGAGGG
TGT

The location of primers and probes is shown relative to the target sequence. The underlined sequence at the 5′ end is the sequence of the excess primer. The underlined sequence at the 3′ end is the sequence that is complementary to the limiting primer. The binding sites of the probes are indicated, with “F-” indicating the probe end having the FAM fluorophore and “-Q” indicating a probe end having a quencher. The Off probe shown is the two variants having a single 5′ quencher. Non-complementary terminal nucleotides are identified (for example, “F-AA” at the 3′ end of the On Probe sequence denotes two non-complementary A's. The two probes in the set hybridize immediately adjacently to one another, that is, there is neither an overlap nor a gap.

LATE-PCR amplifications were performed in triplicate in a 25 μl volume consisting of 1×PCR buffer (Invitrogen, Carlsbad, Calif.), 2 mM MgCl₂, 300 nM dNTPs, 50 nM limiting primers, 1000 nM excess primers, 1.5 units of Platinum Taq DNA Polymerase (Invitrogen, Carlsbad, Calif.), 200 nM of On probes from both probe sets, 500 nM of gyrB Off probe, 500 nM of one version of the 16s Off probe, 1 uM of unlabeled oligo. Three separate mixtures were made with final concentrations of SYBR® Green of 0.12×, 0.24× and 0.48×, respectively. Each of these mixtures was further subdivided into one mixture having the 16s Off probe labeled with a single DABCYL (5′ end), the second having the 16s Off probe labeled with two DABCYLs at both 5′ and 3′ ends, and the third having the 16s Off probe labeled with a single Black Hole quencher (BHQ1).

The thermal profile performed on the Stratagene MxPro 3005P for amplification was as follows: 98° C./3 min for 1 cycle, followed by 98° C./10s-75° C./40s for 60 cycles, followed by 10 min at 75° C., followed by 10 min at 25° C. with a melt starting at 25° C. with 1° C. increments at 30s intervals to 99° C. with fluorescent acquisition (excitation, 492 nm, emission, 516 nm) at each degree. Probe-target hybridizations were analyzed by the melt curve analysis using the first derivative for the temperatures between 25° C. to 95° C.

FIGS. 4A-4F present results obtained for readings in the FAM channel. Fluorescent contours (intensity readings) are presented in FIGS. 4D-4F. Corresponding first derivative curves are presented in FIGS. 4A-4C. As indicated in the figures, FIGS. 4A and 4D are mixtures containing 0.48×SYBR® Green; FIGS. 4B and 4E are mixtures containing 0.24×SYBR® Green; and FIGS. 4C and 4F are mixtures containing 0.12×SYBR® Green. Curves 401, 404, 407, 411, 414 and 417 are mixtures containing the 16s Off Probe with a single DABCYL. Curves 402, 405, 408, 412, 415 and 418 are mixtures containing the 16s Off Probe with two DABCYLs. Curves 403, 406, 409, 413, 416 and 419 are mixtures containing the 16s Off Probe with a single BHQ1.

### Example 5

### Probe Sets Containing on Probes with Dye-Quenching Fluorescent Labels and Unlabeled Probes with Quencher-Only Probes

This example is similar to Example 2 as to drug-susceptible and mutant target strains of the rpoB gene, excess and limiting primers, SYBR® Green dye, and sequences of six probes, but for comparative purposes some of the six quencher-only (“Off”) probes of Example 2 were converted either to On probes by the addition of a terminal Quasar 670 fluorophore (a dye-quenching fluorescent label when used with SYBR® Green dye) or to unlabeled probes by the removal of the quencher moiety. The several modified probe sets were tested for their ability to discriminate a strain of Mycobacterium tuberculosis that is drug susceptible from a strain that is drug resistant for the antibiotic rifampicin due to a point mutation in the rpoB gene target sequence.

A LATE-PCR amplification was performed using a single pair of primers to amplify a 150 base-pair region of the rpoB gene for each strain of Mycobacterium tuberculosis. The amplification provided a 101 base long single-stranded target (Excess Primer Strand), which includes the RRDR region known to contain mutations responsible for drug resistance for rifampicin. Following amplification, each single-stranded nucleic acid target sequence was probed using one of four different sets of six probes that were present in the original amplification reaction mixture.

All four sets of probes spanned the 101 base pairs of the single-stranded nucleic acid target sequence. Combinations of three types of probes were used: quencher probes also labeled with a fluorescent label (“On probes”), quencher-only probes (“Off probes”), and Unlabeled probes. Each On probe was a molecular beacon with a two-nucleotide long stem, and was dual-labeled with a Quasar 670 and a non-fluorescent quencher moiety, BHQ2 (Biosearch Technologies, Novato Calif.) on opposite ends of the oligonucleotide. Each Off probe was terminally labeled with a BHQ2 only. Each probe that did not have a 3′ label had a 3′ nucleotide that was blocked with a carbon linker to prevent extension. In this example different probe sets were used to examine the effect of substituting On probes for some of the Off probes in Example 2 and to examine whether one or more less expensive Unlabeled probes could be substituted for one or more On probes without compromising the capacity of the probe set to distinguish the two strains of M. tuberculosis.

At the end of amplification, probe-target hybridizations were analyzed as a function of temperature. In this example, hybridizations were characterized by the use of melt profile analysis. Reaction components and conditions were as follows:

Limiting Primer:
(SEQ ID No. 6)
5′ CTCCAGCCAGGCACGCTCACGTGACAGACCG
Excess Primer:
(SEQ ID No. 7)
5′CCGGTGGTCGCCGCGATCAAGGAG

### The Off Probe Sequences;

Probe 1
(SEQ ID No. 14)
5′- BHQ2-CTGGTTGGTGCAGAAG-C₃
Probe 3
(SEQ ID No. 16)
5′- BHQ2-CAGCGGGTTGTT-C₃
Probe 6
(SEQ ID No. 19)
5′- ACAGACCGCCGG- BHQ2

### The On Probe Sequences;

Probe 2
(SEQ ID No. 15)
5′-BHQ2 -TCAGGTCCATGAATTGGCTCAGA-Quasar 670
Probe 4
(SEQ ID No. 17)
5′-BHQ2 -ATGCGCTTGTGGATCAACCCCGAT-Quasar 670
Probe 5
(SEQ ID No. 18)
5′-Quasar 670 -AAGCCCCAGCGCCGACAGTCGTT-BHQ2

### The Unlabeled Probe Sequences;

Probe 2-U, the unlabeled version of Probe 2:

| | |
|---|---|
| 5′- TCAGGTCCATGAATTGGCTCAGA- C3 | (SEQ ID No. 15) |

Probe 4-U, the unlabeled version of Probe 4:

| | |
|---|---|
| 5′-ATGCGCTTGTGGATCAACCCCGAT- C₃ | (SEQ ID No. 17) |

Probe 5-U, the unlabeled version of Probe 5:

| | |
|---|---|
| 5′-AAGCCCCAGCGCCGACAGTCGTT-C₃ | (SEQ ID No. 18) |

In the probe sequences, a three-carbon linker, which blocks extension of a probe, is denoted with C₃ while a Black Hole Quencher 2 is denoted with BHQ2. Also in the probe sequences, terminal nucleotides that form a two base-pair molecular beacon stem are underlined.

Target: Strain 8600(L533P)
(SEQ ID No. 10)
5′CCGGTGGTCGCCGCGATCAAGGAGTTCTTCGGCACCAGCCAGCTGAGC
CAATTCATGGACCAGAACAACCCGCTGTCGGGGTTGACCCACAAGCGCCG
ACTGTCGGCGCCGGGGCCCGGCGGTCTGTCACGTGAGCGTGCCTGGCTGG
AG

Target: Strain 24346 (wild type) (same sequence as Target Strain 24609 in Example 2)

```
(SEQ ID No. 8)
                                                        ←  Off #1 →Q
5′CCGGTGGTCGCCGCGATCAAGGAGTTCTTCGGCACCAGCCAGCTGAGC
CAA
                                                                                F-A← On #2
                        ←  Off #3 →Q Q-T-T← On #5
TTCATGGACCAGAACAACCCGCTGTCGGGGTTGACCCACAAGCGCCGACT
GT
      On #2 →Q F-T←  On #4 →T-A-Q
      On #5 →A-A-F
CGGCGCTGGGGCCCGGCGGTCTGTCACGTGAGCGTGCCTGGCTGGAG
                        Q←  Off #6 →
```

The underline in the sequence of strain 8600, which occurs in the Probe 5 (On #5) binding site, denotes the location of the nucleotide change from the drug-sensitive strain 24346. The binding sites of the primers and probes are shown relative to strain 24346. The underlined sequence at the 5′ end is the sequence of the excess primer. The underlined sequence at the 3′ end is the sequence that is complementary to the limiting primer. The binding sites of the six probes are between the arrows (← →) above and below the target sequence. For each probe there is an indication of a quencher (Q). For each On probe there is an indication of a fluorophore (F). Where a probe has one or more non-complementary nucleotides connecting the fluorophore or quencher, those nucleotides are indicated; for example, in On Probe #2 there is a non-complementary 3′-terminal nucleotide A connecting the fluorophore, so there is a designation “F-A←” to indicate that. We note that not all of the probes are perfectly complementary to their binding sites on the target, that is, between the arrows. Off Probe #1 has two mismatches; On Probe #2 has one; and On Probe #4 has one. To aid comparison probe numbering is the same as in Example 2, that is, Probe No. 1 here corresponds to Probe No. 1 in Example 2.

LATE PCR amplifications were carried out in a 25 μl volume consisting of 1×PCR buffer (Invitrogen, Carlsbad, Calif.), 2 mM MgCl₂, 200 nM dNTPs, 50 nM Limiting Primer, 1000 nM Excess Primer, 1.5 units of Platinum Taq DNA Polymerase (Invitrogen, Carlsbad, Calif.), 0.24×SYBR® Green (Invitrogen, Carlsbad, Calif.), 500 nM for probes 1, 3, and 5 and 200 nM for probes 2, 4, and 6 whether labeled or unlabeled. For each strain tested approximately 10,000 genomes equivalents were used. Amplification reactions for each strain were run in triplicate. Reactions were run with differing sets of the six probes. A first probe set included three Off probes (Probes 1,3 and 6) and three On probes (Probes 2,4 and 5) as depicted above. For a second probe set, On probe 2 was converted to an unlabeled oligonucleotide. For a third probe set, On probes 2 and 4 were both converted to unlabeled oligonucleotides. And for a fourth probe set, all three of On probes 2, 4 and 5 were converted to unlabeled oligonucleotides.

The thermal profile for the amplification reaction was as follows: 95° C./3 min for 1 cycle, followed by 60 cycles of 98° C./10s-75° C./40s. This was followed by one cycle of 10 min at 75° C. and 10 min at 25° C. This was followed by a melt with fluorescent acquisition at each degree starting at 25° C. with 1° C. increments at 30s intervals to 97° C. The reactions were done using the Stratagene Mx3005P with excitation and emission for Quasar 670 at 635-665 nm and Fam at 492-516 nm, respectively.

Analysis of the probe target hybridizations following amplification was by melt curve analysis, the results for which are presented as fluorescent signatures (the first derivative) in FIG. 5, panels A-H. Panels in the left column (FIG. 5, panels A, C, E, G) are from readings in the SYBR/FAM channel. Panels in the right column (FIG. 5, panels B, D, F, H) are from readings in the Quasar 670 channel, wherein the non-overlapping Quasar fluorophore was excited directly. FIG. 5, panels A and B are from reactions containing the first set of probes (three Off probes and three On probes, as noted in the legend atop the panels). FIG. 5, panels C and D are from reactions with the second set of probes (three Off probes, two On probes, one unlabeled probe). FIG. 5, panels E and F are from reactions with the third set of probes (three Off probes, one On probe, two unlabeled probes). FIG. 5, panels G and H are from reactions with the fourth set of probes (three Off probes, zero On probes, three unlabeled probes). In FIG. 5 circles 501, 503, 505, 507, 509, 511 identify curves for the wild-type drug sensitive strain; and circles 502, 504, 506, 508, 510, 512 identify curves for the strain having a L533P mutation under On probe 5.

### Example 6

### Double-Stranded Oligonucleotide as a Carrier of SYBR® Green for on-Chip PCR

This example demonstrates the use of SYBR® Green, a DNA binding dye, in PCR amplification carried out in microfluidics device. The method described here uses an excess of a double-stranded DNA oligonucleotide that serves as reservoir of bound dye that is thereby prevented from sticking to the walls of the device and is available to bind to double strands produced during amplification and detection, including the double-stranded product of PCR amplification and probe-target hybrids.

We fabricated a microfluidic device for use in this example. As shown in FIG. 6, the device includes a “chip” 500 plus various controllable inlet and outlet (I/O) ports. Chip 600 comprises two layers of polydimethylsiloxane (“PDMS”), each about 5 mm thick, containing various channels and chambers described below. One layer is a “flow” layer; the other, a “control” layer. Flow channels 611, 612 and well 613 are in the flow layer and communicate with fifty-six reaction chambers 613, also in that layer. Control channels 607, 608 and 610 and reservoir channels 609 are in the control layer, which in FIG. 6 is below the flow layer and bound to a glass slide. Control channel 608 controls whether reaction chambers 613 are open or closed to flow. The two layers are separated by a 15 μm PDMS deformable membrane (not shown). When a positive pressure is applied to a control channel, the membrane at its intersection of the control channel, on one side, and a flow channel, on the other side, deforms and closes that flow channel. I/O port 601 supplies amplification reagents to all the flow channels, when all the reaction chambers, or wells, are to have the same reaction mixture. When different rows of wells 613 are to have different reaction mixtures, however, I/O ports 602 supply the mixtures. I/O port 603 permits regulation of the pressure in control channel 607 by permitting or preventing pressurized fluid to reside in that control supply channel, thereby controlling whether I/O ports 602 are open or closed. Similarly, I/O port 606 permits regulation of the pressure in control supply channel 610 by permitting or preventing pressurized fluid to reside in that control supply channel, thereby controlling whether I/O port 606 is open or closed. I/O port 604 similarly permits regulation of the pressure in control channel 608 to open and close reaction chambers 613. I/O port 605 controls flow to reservoir channels 609.

The width of flow channels 611, 612 is 150 μm. The height of the flow channels is 15-17 μm. The width of control channels 607, 610 is 200 μm. The width of control channels 608 in the direction parallel to reservoir channels 609 is 200 μm. The width of control channels 608 in the direction perpendicular to reservoir channels 609 is 75 μm. The height of the control channels is 30 μm. The width of reservoir channels 609 is 75 μm. The reservoir channel functions to supply water (H₂O) to the layers above it, via diffusion through the PDMS. The diameter of reaction chambers 613 is 300 μm. Their height is 100 μm.

LATE-PCR amplifications were carried out in a PCR well in the device shown in FIG. 6 by placing the device on a flat-surface PCR machine (Advalytix AmpliSpeed Slide Cycler (Beckman Coulter Biomedical GmbH, Munich, Germany)). The device shown in FIG. 6 has rows of eight reaction chambers, which were used to carry out eight replicate reactions under each of the three conditions described below.

### Primers, Probe, and Target Sequence:

Limiting Primer:
(SEQ ID No. 40)
5′-CTGTGCCCTTACATAGTCTAACAGT- 3′
Excess primer:
(SEQ ID No. 41)
5′-ATCGACTTCTTCCACCT -3′
Molecular Beacon Probe:
(SEQ ID No. 41)
5′ -Quasar 670-CGTGCTCCATTGTCCAAACACG-BHQ2-3′

The complementary nucleotides forming the probe stem are underlined.

Target:
5′-CGAGGTCATTGAATACGCACGGCTCCGGGGTATCCGTGTGCTTGCAG
AGTTTGACACTCCTGGCCACACTTTGTCCTGGGGACCAGGTAAGAATGAT
GTCTGGGACCAGAGGGACTCTGCTTGTTATGCTCAGAGTGAAGCTTCAGG
GCACTGGCT
CATGGAAGTGGCATATCCCAGCTTGGTCCTTAGAAGAATGTTTTCC
ATCGACTTCTTCCACCTGGGAATTTAGATAGGAAGAACTCAC
Probe
TTTGGACAATGGAGGCTGCTTCTTACTATTAAAATATGT
ACTGTTAGACTATGTAAGGGCACAGCGC

The underlined nucleotides at the 3′ end of the target are complementary to the limiting primer. The underlined sequence nearest the 5′ end is the sequence of the excess primer. The probe binding site is also indicated.

A series of samples were subjected to LATE-PCR amplification. A first sample contained 500 nM Molecular Beacon Probe and no target sequence, that is, a no-template control (NTC). A second sample contained 500 nM Molecular Beacon Probe and 1,000 copies of target. A third sample contained 500 nM Molecular Beacon Probe and 0.96×SYBR® Green but no target sequence, that is, another NTC. A fourth sample contained 500 nM Molecular Beacon Probe, 0.96×SYBR® Green, and 1,000 copies of target DNA. A fifth sample contained 0.96×SYBR® Green but no target sequence, that is, another NTC. And a sixth sample contained 0.96×SYBR® Green and 1,000 copies of target DNA, but no Molecular Beacon Probe. LATE-PCR amplifications were performed using the microfluidic chip shown in FIG. 6 containing 1×PCR buffer (Invitrogen, Carlsbad, Calif.), 3 mM MgCl2, 200 nM dNTPs, 100 nM limiting primer, 1000 nM excess primer, 2 mg/ml BSA, 150 mM Trehalose, 0.2% Tween-20, and 2U of Platinum Taq DNA polymerase (Invitrogen, Carlsbad, Calif.), and one of the six samples described above.

Each of the six reaction mixtures was filled into a row of reaction chambers of the chip with 10 psi pressure through reagents inlets, I/O ports 102. Each sample was processed in eight replicates. The outlet valves were closed with 25 psi pressure while filling in the samples. Once all the wells were totally filled, control channels 608 were filled with 25 psi pressure to seal the PCR reactions. Then the reservoir was filled with water at 10 psi pressure to compensate for water dissipation in the PCR chambers 613 through the PDMS during PCR cycling. The thermal profile for the amplification reaction was 95 C for 5 minutes followed by 70 cycles of 95 C for 10 sec, 58 C for 20 sec and 72 C for 30 sec. The levels of fluorescence in all reaction chambers were visualized simultaneously using an Olympus IX70 fluorescent microscope. The images were taken in fluorescence channel FITC (excitation wavelength 485/20 nm and emission wavelength 521/20 nm) for the SYBR signal and channel Cy3 (excitation wavelength 560/25 nm and emission wavelength 607/25 nm) for Quas670 Molecular Beacon Probe signal. Fluorescence images were taken before the amplification reaction and following the amplification reaction, after first equilibrating the microfluidics device at room temperature for 10 minutes.

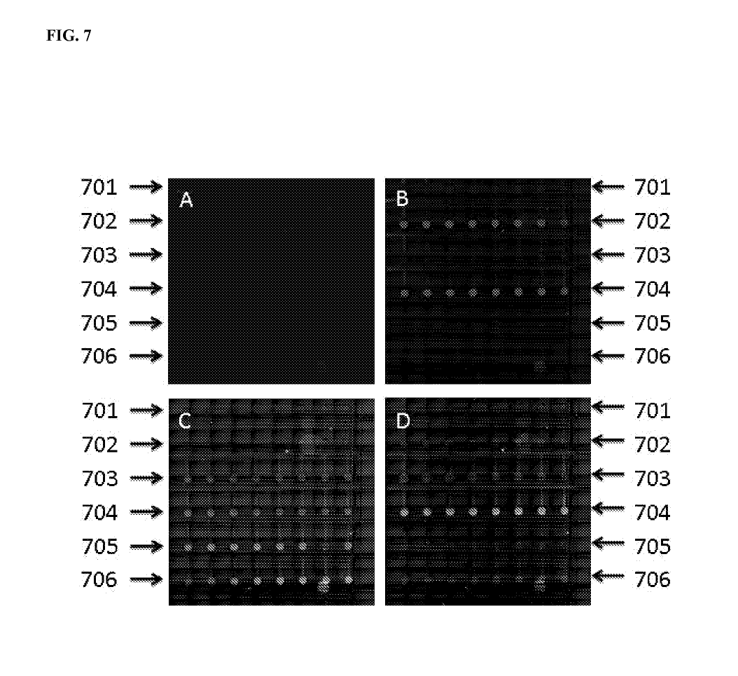

The fluorescence images are shown in FIG. 7, panels A-D. All the images were taken at the same parameter settings and normalized to the same range at image processing. Panel A shows the Molecular Beacon Probe signal before PCR; panel B shows the Molecular Beacon Probe signal after PCR: panel C shows the SYBR signal before PCR; and panel D shows the SYBR signal after PCR. In the images, rows 701 are for the first sample with 500 nM Molecular Beacon Probe but no target sequence (a NTC); rows 702 are for the second sample with 500 nM Molecular Beacon Probe and 1,000 copies of target DNA; rows 703 are for the third sample with 500 nM Molecular Beacon Probe, 0.96×SYBR® Green but no target (another NTC); rows 704 are for the fourth sample with 500 nM Molecular Beacon Probe, 0.96×SYBR, and 1,000 copies of the DNA target sequence; rows 705 are for the fifth sample with 0.96×SYBR® Green but no target sequence (a NTC); rows 706 are for the sixth sample with 0.96×SYBR® Green and 1,000 copies of DNA target sequence but no Molecular Beacon Probe.

As shown in panel A, the Molecular Beacon Probe has no fluorescence before PCR in any sample. In contrast, after PCR in FIG. 2B, both samples with Molecular Beacon Probe and DNA target sequence (rows 702, 704) showed fluorescence, indicating that the PCR worked for both of these samples. For the SYBR® Green signal, panel C shows all of the four samples containing SYBR® Green (rows 703-706) showed more or less SYBR signal before PCR, even the NTC samples (rows 703, 705). This indicates an interaction between SYBR® Green dye and PDMS surfaces, which results in fluorescence. After PCR, panel D shows that the fourth sample (row 704) containing SYBR® Green plus Molecular Beacon as well as target shows fluorescence at a level that is stronger than the fluorescence before PCR. In contrast, the SYBR signal disappeared for the sixth sample, which contained SYBR® Green and target but no Molecular Beacon Probe, as it did for all samples that contained no Molecular Beacon Probe.

1. A homogeneous method for analyzing the nucleic acid content of at least one nucleic acid target sequence contained in copies of at least one nucleic acid target strand as a function of temperature, comprising
a) providing a sample containing copies of said at least one nucleic acid target strand in single-stranded form, said copies having a melting temperature relative to their complementary nucleic acid strands;
b) contacting said single-stranded copies with a double strand DNA binding dye (dsDNA-dye) and a probe set that includes at least one hybridization probe that is complementary to said at least one target sequence and is labeled with at least one non-fluorescent moiety that is a quencher for the dsDNA-dye;
c) subjecting the sample, at multiple temperatures that are below the melting temperature of said copies, to excitation at a wavelength appropriate for stimulating the dye and detecting emission at a wavelength appropriate for detecting emission from the dsDNA-dye; and
d) comparing the detected emissions to corresponding emissions obtained from at least one target sequence whose sequence or nature is known.

2. The method of claim 1 wherein the dsDNA-dye is SYBR® Green.

3. The method of claim 1 wherein the probe set includes an in-situ probe.

4. The method of claim 1 wherein the sample is subjected to melting or annealing over a temperature range spanning the melting temperatures of probe-target hybrids formed in step b).

5. The method of claim 4 wherein the temperature range includes the melting temperature of said copies.

6. The method of claim 4 wherein emission is detected as a fluorescent contour during melting or annealing and comparison comprises comparing fluorescent contours or derivatives thereof (fluorescent signatures), or both.

7. The method of claim 6 wherein the probe set for said at least one target sequence is a multi-probe set that includes at least one dual-labeled fluorescent probe that is quenched when not hybridized but fluorescent when hybridized.

8. The method of claim 7 wherein said at least one dual-labeled probe includes a fluorescent moiety whose emission cannot be distinguished from the dye.

9. The method of claim 8 wherein the dsDNA-dye is SYBR® Green and the fluorescent moiety is FAM.

10. The method of claim 7 wherein said at least one dual-labeled probe includes a fluorescent moiety whose emission is spectrally distinct from the dsDNA-dye's emission, and wherein step c) includes subjecting the sample to excitation at a wavelength appropriate for stimulating the fluorescent moiety and detecting emission at a wavelength appropriate for detecting emission from the fluorescent moiety.

11. The method of claim 10 wherein step d) includes comparing the detected emissions from the fluorescent moiety to corresponding emissions obtained from at least one target sequence whose sequence or nature is known.

12. The method of claim 1 wherein the at least one nucleic acid target sequence is provided by performing a non-symmetric amplification reaction in a reaction mixture comprising said at least one target sequence or its complementary sequence, an excess primer and a limiting primer for said target sequence, dNTPs, a thermally stable DNA polymerase, the dsDNA-dye and the probe set for said at least one target sequence.

13. The method of claim 12 wherein the non-symmetric amplification reaction is a PCR reaction.

14. The method of claim 13 wherein the PCR amplification reaction is a LATE-PCR reaction.

15. The method of claim 13 wherein the dsDNA-dye is SYBR® Green.

16. The method of claim 13 wherein the at least one non-fluorescent quenching moiety is a Black Hole quencher.

17. The method of claim 13 wherein the probe set for said at least one target sequence is a multi-probe set that includes at least one dual-labeled fluorescent probe that is quenched when not hybridized but fluorescent when hybridized.

18. The method of claim 17 wherein said at least one dual-labeled probe includes a fluorescent moiety whose emission cannot be distinguished from the dye.

19. The method of claim 18 wherein the dsDNA-dye is SYBR® Green and the fluorescent moiety is FAM.

20. The method of claim 17 wherein said at least one dual-labeled probe includes a fluorescent moiety whose emission is spectrally distinct from the dsDNA-dye's emission, and wherein step c) includes subjecting the sample to excitation at a wavelength appropriate for stimulating the fluorescent moiety and detecting emission at a wavelength appropriate for detecting emission from the fluorescent moiety.

21. The method of claim 20 wherein step d) includes comparing the detected emissions from the fluorescent moiety to corresponding emissions obtained from at least one target sequence whose sequence or nature is known.

22. The method of claim 13 wherein said at least one target sequence comprises at least two target sequences, and the amplification reaction mixture includes an excess primer, a limiting primer and a probe set for each target sequence.

23. A kit of reagents for amplifying and analyzing at least one target sequence according to the method of claim 12 that comprises an excess primer and a limiting primer for said at least one target sequence, dNTPs, a thermally stable DNA polymerase, a dsDNA-dye and a multi-probe set that includes at least one at least one hybridization probe that is complementary to said at least one target sequence and is labeled only with at least one non-fluorescent moiety that is a quencher for the dsDNA-dye and that includes at least one dual-labeled fluorescent probe hybridization probe that is complementary to said target sequence, that is quenched when not hybridized but fluorescent when hybridized, and that includes a fluorescent moiety whose emission cannot be distinguished from the dye.

24. The kit of claim 23 wherein the dsDNA-dye is SYBR® Green and the fluorescent moiety is the fluorophore FAM.

25. The kit of claim 23 wherein the kit includes the ingredients for an in-situ probe, said ingredients comprising a limiting primer having an extension containing a sequence identical to said at least one target sequence, wherein the complement of said extension is extendable by a DNA polymerase when hybridized to said at least one target sequence.


SEQUENCE LISTING (38 sequences)

SEQ ID NO: 1 - DNA, 37 bp, Artificial Sequence
  Synthetic
agcgcccactcgtagccgtacaggatctcgaggaaac

SEQ ID NO: 2 - DNA, 28 bp, Artificial Sequence
  Synthetic
tcttgggctggaagagctcgtatggcac

SEQ ID NO: 3 - DNA, 139 bp, Mycobacterium tuberculosis
gcttgggctggaagagctcgtatggcaccggaaccggtaaggacgcgatcaccagcggcatcgaggtcgtatggacgaacaccccgacgaaatgggacaacagtttcctcgagatcctgtacggctacgagtgggagct

SEQ ID NO: 4 - DNA, 139 bp, Mycobacterium tuberculosis
gcttgggctggaagagctcgtatggcaccggaaccggtaaggacgcgatcaccaccggcatcgaggtcgtatggacgaacaccccgacgaaatgggacaacagtttcctcgagatcctgtacggctacgagtgggagct

SEQ ID NO: 5 - DNA, 16 bp, Artificial Sequence
  Synthetic
ctcgatgctgctggtg

SEQ ID NO: 6 - DNA, 31 bp, Artificial Sequence
  Synthetic
ctccagccaggcacgctcacgtgacagaccg

SEQ ID NO: 7 - DNA, 24 bp, Artificial Sequence
  Synthetic
ccggtggtcgccgcgatcaaggag

SEQ ID NO: 8 - DNA, 150 bp, Mycobacterium tuberculosis
ccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgcctggctggag

SEQ ID NO: 9 - DNA, 150 bp, Mycobacterium tuberculosis
ccggtggtcgccgcgatcaaggagttcttcggcaccagccagctgagccaattcatggtccagaacaacccgctgtcggggttgacccacaagcgccgactgtcggcgctggggcccggcggtctgtcacgtgagcgtgcctggctggag

SEQ ID NO: 10 - DNA, 150 bp, Mycobacterium tuberculosis
ccggtggtcgccgcgatcaaggagttcttcggcaccagccagctgagccaattcatggaccagaacaacccgctgtcggggttgacccacaagcgccgactgtcggcgccggggcccggcggtctgtcacgtgagcgtgcctggctggag

SEQ ID NO: 11 - DNA, 150 bp, Mycobacterium tuberculosis
ccggtggtcgccgcgatcaaggagttcttcggcaccagccagctgagccaattcatggaccagaacaacccgctgtcggggttgacccgcaagcgccgactgtcggcgctggggcccggcggtctgtcacgtgagcgtgcctggctggag

SEQ ID NO: 12 - DNA, 150 bp, Mycobacterium tuberculosis
ccggtggtcgccgcgatcaaggagttcttcggcaccagccagctgagccaattcatggaccagaacaacccgctgtcggggttgaccctcaagcgccgactgtcggcgctggggcccggcggtctgtcacgtgagcgtgcctggctggag

SEQ ID NO: 13 - DNA, 150 bp, Mycobacterium tuberculosis
ccggtggtcgccgcgatcaaggagttcttcggcaccagccagctgagccaattcatggaccagaacaacccgctgtcggggttgacctacaagcgccgactgtcggcgctggggcccggcggtctgtcacgtgagcgtgcctggctggag

SEQ ID NO: 14 - DNA, 16 bp, Artificial Sequence
  Synthetic
ctggttggtgcagaag

SEQ ID NO: 15 - DNA, 23 bp, Artificial Sequence
  Synthetic
tcaggtccatgaattggctcaga

SEQ ID NO: 16 - DNA, 12 bp, Artificial Sequence
  Synthetic
cagcgggttgtt

SEQ ID NO: 17 - DNA, 24 bp, Artificial Sequence
  Synthetic
atgcgcttgtggatcaaccccgat

SEQ ID NO: 18 - DNA, 23 bp, Artificial Sequence
  Synthetic
aagccccagcgccgacagtcgtt

SEQ ID NO: 19 - DNA, 12 bp, Artificial Sequence
  Synthetic
acagaccgccgg

SEQ ID NO: 20 - DNA, 36 bp, Artificial Sequence
  Synthetic
gtcagcgaaccgcagttcgagggccagaccaagacc

SEQ ID NO: 21 - DNA, 23 bp, Artificial Sequence
  Synthetic
atacgggcttgcgccgaggacac

SEQ ID NO: 22 - DNA, 19 bp, Artificial Sequence
  Synthetic
cgtgtaatgaatagctgcg

SEQ ID NO: 23 - DNA, 20 bp, Artificial Sequence
  Synthetic
aggacgcgaaagtcgttgct

SEQ ID NO: 24 - DNA, 11 bp, Artificial Sequence
  Synthetic
tgaacaaggct

SEQ ID NO: 25 - DNA, 23 bp, Artificial Sequence
  Synthetic
ccactggtttgaagccaacccca

SEQ ID NO: 26 - DNA, 170 bp, Mycobacterium tuberculosis
gtcagcgaaccgcagttcgagggccagaccaagaccaagttgggcaacaccgaggtcaaatcgtttgtgcagaaggtctgtaacgaacagctgacccactggtttgaagccaaccccaccgacgcgaaagtcgttgtgaacaaggctgtgtcctcggcgcaagcccgtat

SEQ ID NO: 27 - DNA, 170 bp, Mycobacterium bovis
gtcagcgaaccgcagttcgagggccagaccaagaccaagttgggcaacaccgaggtcaaatcgtttgtgcagaaggtctgtaatgaacagctgacccactggtttgaagccaaccccaccgactcgaaagtcgttgtgaacaaggctgtgtcctcggcgcaagcccgtat

SEQ ID NO: 28 - DNA, 170 bp, Mycobacterium microti
gtcagcgaaccgcagttcgagggccagaccaagaccaagttgggcaacaccgaggtcaaatcgtttgtgcagaaggtctgtaacgaacagctgacccactggtttgaagccaaccccaccgactcgaaagtcgttgtgaacaaggctgtgtcctcggcgcaagcccgtat

SEQ ID NO: 29 - DNA, 170 bp, Mycobacterium avium
gtgagcgaaccgcagttcgagggccagaccaagaccaaactgggcaacaccgaggtgaagtcgttcgtgcagaaggtgtgcaacgaacagctcacccactggttcgaagccaaccccgcagacgccaaagtcattgtcaacaaggcggtttcgtcagcgcaggcgcgcat

SEQ ID NO: 30 - DNA, 170 bp, Mycobacterium asiaticum
gtcgccgaaccccagttcgagggccagacaaagaccaagctgggcaacaccgaggtcaagtcgttcgtgcagaaggtgtgcaacgaacagctcacccactggttcgaggccaatccgtcggaagccaaaaccgttgtcaacaaggcggtttcgtccgcacaggcccggat

SEQ ID NO: 31 - DNA, 27 bp, Artificial Sequence
  Synthetic
acaccctctcaggccggctacccgtcg

SEQ ID NO: 32 - DNA, 25 bp, Artificial Sequence
  Synthetic
gagtggcgaacgggtgagtaacacg

SEQ ID NO: 33 - DNA, 32 bp, Artificial Sequence
  Synthetic
ttggctcatcccacaccgctaaagtgctttaa

SEQ ID NO: 34 - DNA, 31 bp, Artificial Sequence
  Synthetic
ccaccacaagatatgcgtctcgtgttcctat

SEQ ID NO: 35 - DNA, 211 bp, Mycobacterium tuberculosis
gagtggcgaacgggtgagtaacacgtgggtgatctgccctgcacttcgggataagcctgggaaactgggtctaataccggataggaccacgggatgcatgtcttgtggtggaaagcgctttagcggtgtgggatgagcccgcggcctatcagcttgttggtggggtgacggcctaccaaggcgacgacgggtagccggcctgagagggtgt

SEQ ID NO: 36 - DNA, 209 bp, Mycobacterium asiaticum
gagtggcgaacgggtgagtaacacgtgggtgatctgccctgcacttcgggataagcctgggaaactgggtctaataccggataggaccacgggatgcatgtcctgtggtggaaagcttttgcggtgtgggatgggcccgcggcctatcagcttgttggtggggtgacggcctaccaaggcgacgacgggtagccggcctgagagggtgt

SEQ ID NO: 37 - DNA, 209 bp, Mycobacterium avium
gagtggcgaacgggtgagtaacacgtgggcaatctgccctgcacttcgggataagcctgggaaactgggtctaataccggataggacctcaagacgcatgtcttctggtggaaagcttttgcggtgtgggatgggcccgcggcctatcagcttgttggtggggtgacggcctaccaaggcgacgacgggtagccggcctgagagggtgt

SEQ ID NO: 38 - DNA, 212 bp, Mycobacterium simiae
ctcgagtggcgaacgggtgagtaacacgtgggtaatctgccctgcacttcgggataagcctgggaaactgggtctaataccggataggaccacttggcgcatgccttgtggtggaaagcttttgcggtgtgggatgggcccgcggcctatcagcttgttggtggggtgacggcctaccaaggcgacgacgggtagccgccctgagagggtgt